(19) United States
(12) Patent Application Publication
(10) Pub. No.: US 2024/0158795 A1
(43) Pub. Date: May 16, 2024

(54) Treatment Of Kidney Diseases With A Combination of Angiopoietin Like 3 (ANGPTL3) Inhibitors And Solute Carrier Family 5 Member 2 (SLC5A2) Inhibitors

(71) Applicant: Regeneron Pharmaceuticals, Inc., Tarrytown, NY (US)

(72) Inventors: Mary E. Haas, Tarrytown, NY (US); Kishor Devalaraja-Narashimha, Tarrytown, NY (US); Luca A. Lotta, Tarrytown, NY (US); Lori Morton, Tarrytown, NY (US); Aris Baras, Tarrytown, NY (US); George D. Yancopoulos, Tarrytown, NY (US)

(21) Appl. No.: 18/505,742

(22) Filed: Nov. 9, 2023

Related U.S. Application Data
(60) Provisional application No. 63/424,399, filed on Nov. 10, 2022.

(51) Int. Cl. C12N 15/113; C07K 16/22; C07K 16/24; C12Q 1/6883

(52) U.S. Cl. CPC C12N 15/1136; C07K 16/22; C07K 16/24; C12Q 1/6883; A61K 2039/507; C12N 2310/11

(57) ABSTRACT

The present disclosure provides methods of treating a subject having a kidney disease or preventing a subject from developing a kidney disease by administering an Angiopoietin Like 3 (ANGPTL3) inhibitor and a Solute Carrier Family 5 Member 2 (SLC5A2) inhibitor, and methods of identifying subjects having an increased risk of developing a kidney disease.

### REFERENCE TO SEQUENCE LISTING

This application includes a Sequence Listing filed electronically as an XML file named 381203962SEQ, created on Nov. 9, 2023, with a size of 3,455,297 bytes. The Sequence Listing is incorporated herein by reference.

### FIELD

The present disclosure relates generally to the treatment of subjects having a kidney disease with Angiopoietin Like 3 (ANGPTL3) inhibitors and Solute Carrier Family 5 Member 2 (SLC5A2) inhibitors, and methods of identifying subjects having an increased risk of developing a kidney disease.

### BACKGROUND

In the United States, based on data from the 1999-2006 National Health and Nutrition Examination Survey (NHANES) study, an estimated 11.1 percent (22.4 million) of adults aged 20 or older have chronic kidney disease (CKD) stages 1-3. An additional 0.8 million U.S. adults aged 20 or older have CKD stage 4, and more than 0.3 million have stage 5 CKD and receive hemodialysis. Analyses of NHANES data between 1988-1994 and 1999-2004 suggest that the prevalence of CKD is rising for every CKD stage, but with a particular increase in the prevalence of individuals classified with CKD stage 3. The number of patients with stage 5 CKD requiring dialysis also has increased. It has been estimated that more than 700,000 individuals will have End Stage Renal Disease (ESRD) by 2015. Although CKD can be caused by primary kidney disease (e.g., glomerular diseases, tubulointerstitial diseases, obstruction, and polycystic kidney disease), in the vast majority of patients with CKD, the kidney damage is associated with other medical conditions such as diabetes and hypertension. In 2008, excluding those with ESRD, 48 percent of Medicare patients with CKD had diabetes, 91 percent had hypertension, and 46 percent had atherosclerotic heart disease. Other risk factors for CKD include age, obesity, family history, and ethnicity. CKD has been associated with numerous adverse health outcomes.

A Glomerular Filtration Rate (GFR) of 90 mL/min or higher (Stage 1) is normal in most healthy people. Usually, few symptoms are present at this stage of CKD. A GFR of 60-89 mL/min (Stage 2) may for some patients, such as the elderly or infants, be normal if no kidney damage is present. A GFR between 60-89 mL/min for three months or longer along with kidney damage is a sign of early CKD. Usually, few symptoms are present at this stage. A GFR between 30-59 mL/min (Stage 3) for a patient is indicative of moderate CKD, and are more likely to develop anemia, early bone disease or high blood pressure, and may desire to see a nephrologist. A GFR between 15-29 mL/min (Stage 4) indicates that the patient has severe CKD and will likely need dialysis or a kidney transplant in the future. A GFR of 15 mL/min or less (Stage 5) indicates that the patient has chronic CKD and have ESRD. The kidneys have lost almost all ability to function effectively at this stage. They will need dialysis or a kidney transplant to live.

The ANGPTL3 gene encodes a member of a family of secreted proteins that function in angiogenesis. The encoded protein, which is expressed predominantly in the liver, is further processed into an N-terminal coiled-coil domain-containing chain and a C-terminal fibrinogen chain. The N-terminal chain is important for lipid metabolism, while the C-terminal chain may be involved in angiogenesis. Mutations in this gene cause familial hypobetalipoproteinemia type 2.

The SLC5A2 gene encodes a low affinity, high capacity Na(+)/glucose cotransporter, which is located in the early proximal convoluted tubule segment S1, and has a Na(+)-to-glucose coupling ratio of 1:1. It is the major reabsorptive mechanism for D-glucose in the kidney.

### SUMMARY

The present disclosure provides methods of treating a subject having a kidney disease or at risk of developing a kidney disease, the methods comprising administering an ANGPTL3 inhibitor and an SLC5A2 inhibitor to the subject.

The present disclosure also provides methods of treating a subject with a kidney disease therapeutic agent, wherein the subject has a kidney disease or is at risk of developing a kidney disease, by administering a kidney disease therapeutic agent, the methods comprising: determining whether the subject has an ANGPTL3 variant nucleic acid molecule and whether the subject has an SLC5A2 variant nucleic acid molecule, by: obtaining or having obtained a biological sample from the subject; and performing or having performed a sequence analysis on the biological sample to determine if the subject has a genotype comprising the ANGPTL3 variant nucleic acid molecule and the SLC5A2 variant nucleic acid molecule; and administering or continuing to administer the kidney disease therapeutic agent to a subject that is homozygous for both the ANGPTL3 variant nucleic acid molecule and the SLC5A2 variant nucleic acid molecule; or administering or continuing to administer the kidney disease therapeutic agent and/or an ANGPTL3 inhibitor and an SLC5A2 inhibitor to a subject that is: i) heterozygous for both the ANGPTL3 variant nucleic acid molecule and the SLC5A2 variant nucleic acid molecule, ii) heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule, or iii) ANGPTL3 reference and SLC5A2 reference; wherein the presence of a genotype having the ANGPTL3 variant nucleic acid molecule and the SLC5A2 variant nucleic acid molecule indicates the subject has a decreased risk of developing a kidney disease.

The present disclosure also provides methods of identifying a subject having an increased risk of developing a kidney disease, the methods comprising: determining or having determined the presence or absence of an ANGPTL3 variant nucleic acid molecule and determining or having determined the presence or absence of an SLC5A2 variant nucleic acid molecule in a biological sample obtained from the subject; wherein: when the subject is ANGPTL3 reference and SLC5A2 reference, then the subject has an increased risk of developing a kidney disease; and when the subject is heterozygous or homozygous for the ANGPTL3 variant nucleic acid molecule and heterozygous or homozygous for the SLC5A2 variant nucleic acid molecule, or when the subject is heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule, then the subject has a decreased risk of developing a kidney disease.

The present disclosure also provides kidney disease therapeutic agents for use in the treatment or prevention of a kidney disease in a subject having an ANGPTL3 variant nucleic acid molecule and having an SLC5A2 variant nucleic acid molecule.

The present disclosure also provides ANGPTL3 inhibitors and SLC5A2 inhibitors for use in the treatment or prevention of a kidney disease in a subject that is: i) ANGPTL3 reference or heterozygous for the ANGPTL3 variant nucleic acid molecule, ii) SLC5A2 reference or heterozygous for the SLC5A2 variant nucleic acid molecule, and iii) heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting of” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that rare ANGPTL3 variant nucleic acid molecules and rare SLC5A2 variant nucleic acid molecules have independent effects on decreased risk of developing a kidney disease. Both the rare ANGPTL3 variant nucleic acid molecules and the rare SLC5A2 variant nucleic acid molecules result in an increased estimated glomerular filtration rate (eGFR). It would have been expected that two genes that each result in increased eGFR could operate through the same mechanism/pathway. If this were the case, the effect of variants in one gene would be expected to depend on whether variants in the second gene were also present. If this were the case, the effect of one gene would have been expected to have a different magnitude of effect when in combination with the effect of the other gene. Surprisingly, the increased eGFR associated with rare ANGPTL3 variant nucleic acid molecules was instead independent of the increased eGFR associated with rare SLC5A2 variant nucleic acid molecules. Therefore, subjects that are ANGPTL3 reference or heterozygous for an ANGPTL3 variant nucleic acid molecule and that are SLC5A2 reference or heterozygous for an SLC5A2 variant nucleic acid molecule have the benefit of being treated with inhibitors of two different biological pathways (e.g., with an ANGPTL3 inhibitor and an SLC5A2 inhibitor) such that a kidney disease is inhibited or prevented, the symptoms thereof are reduced or prevented, and/or development of symptoms is repressed or prevented. It is also believed that such subjects having a kidney disease may further be treated with kidney disease therapeutic agents.

For purposes of the present disclosure, any particular subject, such as a human, can be categorized as having one of three ANGPTL3 genotypes: i) ANGPTL3 reference; ii) heterozygous for an ANGPTL3 variant nucleic acid molecule; or iii) homozygous for an ANGPTL3 variant nucleic acid molecule. A subject is ANGPTL3 reference when the subject does not have a copy of an ANGPTL3 variant nucleic acid molecule. A subject is heterozygous for an ANGPTL3 variant nucleic acid molecule when the subject has a single copy of an ANGPTL3 variant nucleic acid molecule.

In any of the embodiments described herein, the ANGPTL3 variant nucleic acid molecule can be any nucleic acid molecule (such as, a genomic nucleic acid molecule, an mRNA molecule, or a cDNA molecule produced from an mRNA molecule) encoding an ANGPTL3 variant polypeptide having a partial loss-of-function, a complete loss-of-function, a predicted partial loss-of-function, or a predicted complete loss-of-function. A subject who has an ANGPTL3 polypeptide having a partial loss-of-function (or predicted partial loss-of-function) is hypomorphic for ANGPTL3. In some embodiments, the ANGPTL3 variant nucleic acid molecule results in decreased or aberrant expression or activity of ANGPTL3 mRNA or polypeptide. In some embodiments, the ANGPTL3 variant nucleic acid molecule is associated with a reduced in vitro response to ANGPTL3 ligands compared with reference ANGPTL3. In some embodiments, the ANGPTL3 variant nucleic acid molecule is a splice-site variant, a stop-gain variant, a start-loss variant, a stop-loss variant, a frameshift variant, an in-frame indel variant, or a variant that encodes a truncated ANGPTL3 variant polypeptide. In some embodiments, the ANGPTL3 variant nucleic acid molecule is a missense variant nucleic acid molecule. In some embodiments, the ANGPTL3 variant nucleic acid molecule comprises a single nucleotide polymorphism (SNP). In some embodiments, the ANGPTL3 variant nucleic acid molecule comprises a variation in a coding region. In some embodiments, the ANGPTL3 variant nucleic acid molecule results or is predicted to result in a premature truncation of an ANGPTL3 polypeptide compared to the reference ANGPTL3. In some embodiments, the ANGPTL3 variant nucleic acid molecule is a variant that is predicted to be damaging to the protein function (and hence, in this case, protective to the human) by in vitro prediction algorithms such as Polyphen, SIFT, or similar algorithms. In some embodiments, the ANGPTL3 variant nucleic acid molecule is a variant that causes or is predicted to cause a nonsynonymous amino acid substitution in an ANGPTL3 nucleic acid molecule and whose allele frequency is less than 1/100 alleles in the population from which the subject is selected. In some embodiments, the ANGPTL3 variant nucleic acid molecule is any rare missense variant (allele frequency<0.1%; or 1 in 1,000 alleles), or any splice-site, stop-gain, start-loss, stop-loss, frameshift, or in-frame indel, or other frameshift ANGPTL3 variant.

For purposes of the present disclosure, any particular subject, such as a human, can be categorized as having one of three SLC5A2 genotypes: i) SLC5A2 reference; ii) heterozygous for an SLC5A2 variant nucleic acid molecule; or iii) homozygous for an SLC5A2 variant nucleic acid molecule. A subject is SLC5A2 reference when the subject does not have a copy of an SLC5A2 variant nucleic acid molecule. A subject is heterozygous for an SLC5A2 variant nucleic acid molecule when the subject has a single copy of an SLC5A2 variant nucleic acid molecule.

In some embodiments, the subject is heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule.

In any of the embodiments described herein, the SLC5A2 variant nucleic acid molecule can be any nucleic acid molecule (such as, a genomic nucleic acid molecule, an mRNA molecule, or a cDNA molecule produced from an mRNA molecule) encoding an SLC5A2 variant polypeptide having a partial loss-of-function, a complete loss-of-function, a predicted partial loss-of-function, or a predicted complete loss-of-function. A subject who has an SLC5A2 polypeptide having a partial loss-of-function (or predicted partial loss-of-function) is hypomorphic for SLC5A2. In some embodiments, the SLC5A2 variant nucleic acid molecule results in decreased or aberrant expression or activity of SLC5A2 mRNA or polypeptide. In some embodiments, the SLC5A2 variant nucleic acid molecule is associated with a reduced in vitro response to SLC5A2 ligands compared with reference SLC5A2. In some embodiments, the SLC5A2 variant nucleic acid molecule is a splice-site variant, a stop-gain variant, a start-loss variant, a stop-loss variant, a frameshift variant, an in-frame indel variant, or a variant that encodes a truncated SLC5A2 variant polypeptide. In some embodiments, the SLC5A2 variant nucleic acid molecule is a missense variant nucleic acid molecule. In some embodiments, the SLC5A2 variant nucleic acid molecule comprises a single nucleotide polymorphism (SNP). In some embodiments, the SLC5A2 variant nucleic acid molecule comprises a variation in a coding region. In some embodiments, the SLC5A2 variant nucleic acid molecule results or is predicted to result in a premature truncation of an SLC5A2 polypeptide compared to the reference SLC5A2. In some embodiments, the SLC5A2 variant nucleic acid molecule is a variant that is predicted to be damaging to the protein function (and hence, in this case, protective to the human) by in vitro prediction algorithms such as Polyphen, SIFT, or similar algorithms. In some embodiments, the SLC5A2 variant nucleic acid molecule is a variant that causes or is predicted to cause a nonsynonymous amino acid substitution in an SLC5A2 nucleic acid molecule and whose allele frequency is less than 1/100 alleles in the population from which the subject is selected. In some embodiments, the SLC5A2 variant nucleic acid molecule is any rare missense variant (allele frequency<0.1%; or 1 in 1,000 alleles), or any splice-site, stop-gain, start-loss, stop-loss, frameshift, or in-frame indel, or other frameshift SLC5A2 variant.

For subjects that are genotyped or determined to be ANGPTL3 reference and SLC5A2 reference, such subjects have an increased risk of developing a kidney disease. For subjects that are genotyped or determined to be: i) either ANGPTL3 reference or heterozygous for an ANGPTL3 variant nucleic acid molecule, and ii) either SLC5A2 reference or heterozygous for an SLC5A2 variant nucleic acid molecule, such subjects can be treated with an ANGPTL3 inhibitor and an SLC5A2 inhibitor.

In any of the embodiments described herein, the subject in whom a kidney disease is prevented by administering the ANGPTL3 inhibitor and SLC5A2 inhibitor can be anyone at risk for developing a kidney disease including, but not limited to, subjects with a genetic predisposition for developing a kidney disease. Additional risk factors include, but are not limited to, diabetes, hypertension, obesity, excessive salt intake, age, smoking, excessive alcohol consumption, heavy metal exposure, hyperlipidemia, and the presence of autoimmune diseases. In addition, in some embodiments, any subject can be at risk of developing a kidney disease. In some embodiments, administering an ANGPTL3 inhibitor and an SLC5A2 inhibitor can be carried out to prevent development of an additional kidney disease in a subject who has already had a kidney disease.

In any of the embodiments described herein, the ANGPTL3 polypeptide can be any ANGPTL3 polypeptide having a partial loss-of-function, a complete loss-of-function, a predicted partial loss-of-function, or a predicted complete loss-of-function. In any of the embodiments described herein, the SLC5A2 polypeptide can be any SLC5A2 polypeptide having a partial loss-of-function, a complete loss-of-function, a predicted partial loss-of-function, or a predicted complete loss-of-function.

In any of the embodiments described herein, the ANGPTL3 variant nucleic acid molecule (such as genomic nucleic acid molecules, mRNA molecules produced therefrom, or cDNA molecules produced from the mRNA molecules) can include variations at positions 62,597,520-62,606,313 of chromosome 1 using the nucleotide sequence of the ANGPTL3 reference genomic nucleic acid molecule (see, ENST00000371129.4 annotated in the in the Ensembl database (URL: world wide web at “useast.ensembl.org/Homo_sapiens/Transcript/Summary?db=core;g=ENSG00000132855;r=1:62597520-62606313;t=ENST00000371129”)) as a reference sequence. The sequence provided in ENST00000371129.4 for the ANGPTL3 genomic nucleic acid molecule is only an exemplary sequence. Other sequences for the ANGPTL3 genomic nucleic acid molecule are also possible. Exemplary ANGPTL3 variant nucleic acid molecules include, but are not limited to those recited in Table 6.

In any of the embodiments described herein, the SLC5A2 variant nucleic acid molecule (such as genomic nucleic acid molecules, mRNA molecules produced therefrom, or cDNA molecules produced from the mRNA molecules) can include variations at positions 31,483,123-31,490,769 of chromosome 16 using the nucleotide sequence of the SLC5A2 reference genomic nucleic acid molecule (see, ENST00000330498.4 annotated in the in the Ensembl database (URL: world wide web at “useast.ensembl.org/Homo_sapiens/Gene/Summary?g=ENSG00000140675;r=16:31483002-31490860;transcript=ENST00000330498.4”)) as a reference sequence. The sequence provided in ENST00000330498.4 for the SLC5A2 genomic nucleic acid molecule is only an exemplary sequence. Other sequences for the SLC5A2 genomic nucleic acid molecule are also possible. Exemplary SLC5A2 variant nucleic acid molecules include, but are not limited to those recited in 6.

Any one or more (i.e., any combination) of the ANGPTL3 variant nucleic acid molecules and SLC5A2 variant nucleic acid molecules described herein can be used within any of the methods described herein to determine whether a subject has an increased or decreased risk of developing a kidney disease. The combinations of particular variants can form a mask used for statistical analysis of the particular correlation of ANGPTL3 and SLC5A2 and an increased or decreased risk of developing a kidney disease. In some embodiments, the mask used for statistical analysis of the particular correlation of ANGPTL3 and SLC5A2 and an increased or decreased risk of developing a kidney disease can exclude any one or more of these ANGPTL3 variant nucleic acid molecules and/or SLC5A2 variant nucleic acid molecules described herein.

In any of the embodiments described herein, the subject can have a kidney disease. In any of the embodiments described herein, the subject can be at risk of developing a kidney disease. In any of the embodiments described herein, the kidney disease is chronic kidney disease, diabetic kidney disease, a kidney stone, chronic glomerulonephritis, nephronophthisis, chronic interstitial nephritis, and/or nephrosclerosis. In some embodiments, the kidney disease is chronic kidney disease. In some embodiments, the kidney disease is diabetic kidney disease. In some embodiments, the kidney disease is a kidney stone. In some embodiments, the kidney disease is chronic glomerulonephritis. In some embodiments, the kidney disease is nephronophthisis. In some embodiments, the kidney disease is chronic interstitial nephritis. In some embodiments, the kidney disease is nephrosclerosis.

Other kidney disease include, but are not limited to, acquired cystic disease, acute (postinfectious) glomerulonephritis, acute infectious interstitial nephritis, acute interstitial nephritis, acute pyelonephritis, acute renal failure, acute transplant failure, acute tubular necrosis, adult polycystic kidney disease, AL amyloid, analgesic nephrosis, ANCA-associated vasculitis, anti-glomerular basement membrane disease (Goodpasture's Syndrome), antibody-mediated kidney graft rejection, asymptomatic hematuria, asymptomatic proteinuria, atypical hemolytic uremic syndrome, autosomal dominant polycystic kidney disease, autosomal recessive polycystic kidney disease, BK virus-associated nephropathy, Bence Jones cast nephrosis, benign familial hematuria, benign nephrosclerosis and atheromatous embolization, bilateral cortical necrosis, C3 glomerulonephritis, cardiac surgery associated acute kidney injury, chronic allograft nephropathy, chronic glomerulonephritis, chronic interstitial nephritis, chronic pyelonephritis, chronic renal failure, chronic transplant failure, circulating immune complex nephritis, contrast-induced nephropathy, crescentic glomerulonephritis, cryoglobulinemia, cystic renal dysplasia, delayed graft function, dense deposit disease, diabetic glomerulosclerosis, diabetic nephropathy, dialysis cystic disease, drug induced (allergic) acute interstitial nephritis, ectopic kidney, eosinophilic granulomatosis with polyangiitis, Fabry's disease, familial juvenile nephronophthisis-medullary cystic disease complex, focal segmental glomerulosclerosis (segmental hyalinosis), glomerulocystic disease, glomerulonephritis, glomerulonephritis associated with bacterial endocarditis, glomerulosclerosis, granulomatosis with polyangiitis, hemolytic-uremic syndrome, Henoch-Schonlein purpura, hepatitis-associated glomerulonephritis, hereditary nephritis (Alport syndrome), Human Immunodeficiency Virus-associated nephropathy, horseshoe kidney, hydronephrosis, hyperoxaluria, hypertensive nephropathy, IgA nephropathy, infantile polycystic kidney disease, ischemic acute tubular necrosis, light-chain deposit disease, lupus nephritis, malignant nephrosclerosis, medullary cystic disease, membranoproliferative (mesangiocapillary) glomerulonephritis, membranous glomerulonephritis, membranous nephropathy, mesangial proliferative glomerulonephritis (includes Berger's Disease), microscopic polyangiitis, minimal change glomerular disease, nephritic syndrome, nephroblastoma (Wilms tumor), nephronophthisis (medullary cystic disease complex), pigment nephropathy, plasma cell dyscrasias (monoclonal immunoglobulin-induced renal damage), polyarteritis nodosa, polycystic kidney disease, proteinuria, pyelonephritis, rapidly progressive (crescentic) glomerulonephritis, renal agenesis, renal amyloidosis, renal cell carcinoma, renal dysgenesis, renal dysplasia, renal hypoplasia, renal infection, renal osteodystrophy, renal stones (urolithiasis), renal tubular acidosis, renal vasculitis, renovascular hypertension, scleroderma (progressive systemic sclerosis), secondary acquired glomerulonephritis, sepsis-associated acute kidney injury, simple renal cysts, systemic lupus erythematosus, T-cell-mediated kidney graft rejection, thin basement membrane nephropathy, thrombotic microangiopathy, thrombotic thrombocytopenic purpura, toxic acute tubular necrosis, tubular defects, tubulointerstitial disease in multiple myeloma, urate nephropathy, urinary obstruction, and vasculitis.

Symptoms of chronic kidney disease include, but are not limited to, nausea, vomiting, loss of appetite, fatigue and weakness, sleep problems, changes urination volume, decreased mental sharpness, muscle twitches and cramps, swelling of feet and ankles, persistent itching, chest pain, fluid build-up around the lining of the heart, shortness of breath, fluid build-up in the lungs, and high blood pressure (hypertension) that's difficult to control.

Symptoms of a kidney stone include, but are not limited to, severe, sharp pain in the side and back, below the ribs, pain that radiates to the lower abdomen and groin, pain that comes in waves and fluctuates in intensity, pain or burning sensation while urinating, pink, red or brown urine, cloudy or foul-smelling urine, a persistent need to urinate, urinating more often than usual or urinating in small amounts, nausea and vomiting, and fever and chills if an infection is present.

Symptoms of chronic glomerulonephritis include, but are not limited to, pink or cola-colored urine from red blood cells in your urine (hematuria), foamy urine due to excess protein (proteinuria), high blood pressure (hypertension), and fluid retention (edema) with swelling evident in the face, hands, feet, and abdomen.

Symptoms of nephronophthisis include, but are not limited to, increased urine production (polyuria), excessive thirst (polydipsia), general weakness, and extreme tiredness (fatigue).

Symptoms of chronic interstitial nephritis include, but are not limited to, blood in the urine, fever, increased or decreased urine output, mental status changes (drowsiness, confusion, coma), nausea, vomiting, rash, swelling of any area of body, and weight gain (from retaining fluid).

Symptoms of nephrosclerosis include, but are not limited to, impaired vision, blood in the urine, loss of weight, and the accumulation of urea and other nitrogenous waste products in the blood, a condition known as uremia.

The present disclosure provides methods of treating a subject having a kidney disease or at risk of developing a kidney disease, the method comprising administering an ANGPTL3 inhibitor and an SLC5A2 inhibitor to the subject. In some embodiments, the kidney disease is chronic kidney disease. In some embodiments, the kidney disease is diabetic kidney disease. In some embodiments, the kidney disease is a kidney stone. In some embodiments, the kidney disease is chronic glomerulonephritis. In some embodiments, the kidney disease is nephronophthisis. In some embodiments, the kidney disease is chronic interstitial nephritis. In some embodiments, the kidney disease is nephrosclerosis.

In some embodiments, the ANGPTL3 inhibitor comprises an inhibitory nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of an ANGPTL3 nucleic acid molecule. In some embodiments, the antisense RNA, siRNA, or shRNA hybridizes to a sequence within an ANGPTL3 genomic nucleic acid molecule or mRNA molecule and decreases expression of the ANGPTL3 polypeptide in a cell in the subject. In some embodiments, the ANGPTL3 inhibitor comprises an antisense molecule that hybridizes to an ANGPTL3 genomic nucleic acid molecule or mRNA molecule and decreases expression of the ANGPTL3 polypeptide in a cell in the subject. In some embodiments, the ANGPTL3 inhibitor comprises an siRNA that hybridizes to an ANGPTL3 genomic nucleic acid molecule or mRNA molecule and decreases expression of the ANGPTL3 polypeptide in a cell in the subject. In some embodiments, the ANGPTL3 inhibitor comprises an shRNA that hybridizes to an ANGPTL3 genomic nucleic acid molecule or mRNA molecule and decreases expression of the ANGPTL3 polypeptide in a cell in the subject.

In some embodiments, the ANGPTL3 antisense nucleic acid molecules comprise or consist of any of the nucleotide sequences represented by SEQ ID NOs: 1-325. In some embodiments, the ANGPTL3 siRNA molecules comprise or consist of any of the nucleotide sequences (sense and antisense strands presented one after the other) represented by SEQ ID NOs: 326-1189 (e.g., the sense strand is, for example, SEQ ID NO:326 and the corresponding antisense strand is SEQ ID NO:327; the sense strand is, for example, SEQ ID NO:1188 and the corresponding antisense strand is SEQ ID NO:1189; etc.).

In some embodiments, the siRNA molecules comprise or consist of the nucleotide sequences (sense and antisense strands) recited in U.S. Pat. No. 10,995,335 and PCT Publication No. WO 2019/055633, which are incorporated herein by reference in their entirety.

In some embodiments, the siRNA molecules comprise or consist of the nucleotide sequences (sense and antisense strands) recited in U.S. Pat. No. 10,875,884 and PCT Publication Nos. WO 2015/168589, WO 2015/100394, and WO 2011/085271, which are incorporated herein by reference in their entirety.

In some embodiments, the siRNA molecules comprise or consist of the nucleotide sequences (sense and antisense strands) recited in U.S. Pat. Nos. 10,570,393 and 10,337,010, and PCT Publication Nos. WO 2016/168286 and WO 2012/177784, which are incorporated herein by reference in their entirety.

In some embodiments, the ANGPTL3 inhibitor comprises a nuclease agent that induces one or more nicks or double-strand breaks at a recognition sequence(s) or a DNA-binding protein that binds to a recognition sequence within an ANGPTL3 genomic nucleic acid molecule. The recognition sequence can be located within a coding region of the ANGPTL3 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ANGPTL3 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an ANGPTL3 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of ANGPTL3 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an ANGPTL3 genomic nucleic acid molecule or it can be a nickase that creates a single-strand break in an ANGPTL3 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be joined or fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of ANGPTL3 genomic nucleic acid molecules can be generated by contacting a cell with a Cas protein and one or more gRNAs that hybridize to one or more gRNA recognition sequences within a target genomic locus in the ANGPTL3 genomic nucleic acid molecule. The gRNA recognition sequence can include or be proximate to the start codon of an ANGPTL3 genomic nucleic acid molecule or the stop codon of an ANGPTL3 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an ANGPTL3 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an ANGPTL3 genomic nucleic acid molecule. An exemplary gRNA is a gRNA effective to direct a Cas enzyme to bind to or cleave an ANGPTL3 genomic nucleic acid molecule, wherein the gRNA comprises a DNA-targeting segment that hybridizes to a gRNA recognition sequence within the ANGPTL3 genomic nucleic acid molecule. Exemplary gRNAs comprise a DNA-targeting segment that hybridizes to a gRNA recognition sequence present within an ANGPTL3 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the target ANGPTL3 genomic nucleic acid molecule. The Cas protein can cleave the nucleic acid molecule at a site within or outside of the nucleic acid sequence present in the target ANGPTL3 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ANGPTL3 genomic nucleic acid molecule to which a DNA-targeting segment of a gRNA will bind.

Such methods can result, for example, in an ANGPTL3 genomic nucleic acid molecule in which a region of the ANGPTL3 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ANGPTL3 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the ANGPTL3 inhibitor is a small molecule. In some embodiments, the ANGPTL3 inhibitor is (12mer-)heparin (Gunn et al., J. Biol. Chem., 2021, 296, 1-12) or CAT-2003 (Liu et al., Arteriosclerosis, Thrombosis, and Vascular Biology, 2014, 34, A237). In some embodiments, the ANGPTL3 inhibitor is a vaccine. In some embodiments, the vaccine comprises a peptide corresponding to the LPL inhibitory domain of ANGPTL3. In some embodiments, the vaccine comprises a peptide having an amino acid sequence comprising amino acids 32 to 41 of ANGPTL3 (i.e., EPKSRFAMLD; SEQ ID NO:3738) (see, Fukami et al., Cell Reports Med., 2021, 100446).

In some embodiments, the ANGPTL3 inhibitor is an antibody, or antigen-binding fragment thereof. In some embodiments, the antibody, or antigen-binding fragment thereof, binds specifically to human ANGPTL3. Exemplary antibodies, and fragments thereof, are disclosed in PCT Publication WO 2020/243031, which is incorporated herein by reference in its entirety.

In some embodiments, the antibody is a fully human monoclonal antibody (mAb), or antigen-binding fragment thereof, that specifically binds and neutralizes, inhibits, blocks, abrogates, reduces, or interferes with, at least one activity of ANGTPL3, in particular, human ANGPTL3 (SEQ ID NO:3739). The activity of ANGPTL3 that can be neutralized, inhibited, blocked, abrogated, reduced or interfered with, by the antibodies or fragments thereof of the present disclosure, includes, but is not limited to, inhibition of LPL activity, induction of angiogenesis, and the like. In some embodiments, an antibody or fragment thereof can neutralize, inhibit, block, abrogate, reduce, or interfere with, an activity of ANGPTL3 by binding to an epitope of ANGPTL3 that is directly involved in the targeted activity of ANGPTL3. In some embodiments, an antibody or fragment thereof can neutralize, inhibit, block, abrogate, reduce, or interfere with, an activity of ANGPTL3 by binding to an epitope of ANGPTL3 that is not directly involved in the targeted activity of ANGPTL3, but the antibody or fragment binding thereto sterically or conformationally inhibits, blocks, abrogates, reduces, or interferes with, the targeted activity of ANGPTL3. In some embodiments, an antibody or fragment thereof binds to an epitope of ANGPTL3 that is not directly involved in the targeted activity (e.g., inhibiting LPL activity, inducing angiogenesis, and the like) of ANGPTL3 (i.e., a non-blocking antibody), but the antibody or fragment binding thereto results in the enhancement of the clearance of ANGPTL3 from the circulation, compared to the clearance of ANGPTL3 in the absence of the antibody or fragment thereof, thereby indirectly inhibiting, blocking, abrogating, reducing, or interfering with, an activity of ANGPTL3. Clearance of ANGPTL3 from the circulation can be particularly enhanced by combining two or more different non-blocking antibodies that do not compete with one another for specific binding to ANGPTL3.

The antibodies (Abs) can be full-length (for example, an IgGl or IgG4 antibody) or may comprise only an antigen-binding portion (for example, a Fab, F(ab′)₂ or scFv fragment), and may be modified to affect functionality, e.g., to eliminate residual effector functions (Reddy et al., J. Immunol., 2000, 164, 1925-1933).

In some embodiments, the antibody or antigen-binding fragment of an antibody comprises a heavy chain variable region (HCVR) selected from the group consisting of SEQ ID NO:3740, 3741, 3742, 3743, 3744, 3745, 3746, 3747, 3748, 3749, and 3750, or a substantially similar sequence thereof having at least 90%, at least 95%, at least 98% or at least 99% sequence identity. In some embodiments, the antibody or antigen-binding fragment thereof comprises a HCVR having an amino acid sequence selected from the group consisting of SEQ ID NO:3740, 3741, 3742, 3744, 3745, 3747, and 3750. In some embodiments, the antibody or an antigen-binding fragment thereof comprises a HCVR having an amino acid sequence of SEQ ID NO:3744.

In some embodiments, an antibody or antigen-binding fragment of an antibody comprises a light chain variable region (LCVR) selected from the group consisting of SEQ ID NO:3751, 3752, 3753, 3754, 3755, 3756, 3757, 3758, 3759, 3760, and 3761, or a substantially similar sequence thereof having at least 90%, at least 95%, at least 98% or at least 99% sequence identity. In another embodiment, the antibody or antigen-binding portion of an antibody comprises a LCVR having an amino acid sequence selected from the group consisting of SEQ ID NO:3751, 3752, 3753, 3755, 3756, 3758, and 3761. In some embodiments, the antibody or antigen-binding portion of an antibody comprises a LCVR having an amino acid sequence of SEQ ID NO:3755.

In some embodiments, the antibody or fragment thereof comprises a HCVR and LCVR sequence pair (HCVR/LCVR) selected from the group consisting of SEQ ID NO:3740/3751, 3741/3752, 3742/3753, 3743/3754, 3744/3755, 3745/3756, 3746/3757, 3747/3758, 3748/3759, 3749/3760, and 3750/3761. In some embodiments, the antibody or fragment thereof comprises a HCVR and LCVR sequence pair selected from the group consisting of SEQ ID NO:3740/3751, 3741/3752, 3742/3753, 3744/3755, 3745/3756, 3747/3758, and 3750/3761. In some embodiments, the antibody or fragment thereof comprises a HCVR and LCVR sequence pair of SEQ ID NO:3744/3755.

In some embodiments, the antibody or antigen-binding fragment thereof comprises a heavy chain complementarity determining region 3 (HCDR3) amino acid sequence selected from the group consisting of SEQ ID NO:3762, 3763, 3764, 3765, 3766, 3767, 3768, 3769, 3770, 3771, and 3772, or a substantially similar sequence thereof having at least 90%, at least 95%, at least 98% or at least 99% sequence identity; and a light chain CDR3 (LCDR3) amino acid sequence selected from the group consisting of SEQ ID NO:3773, 3774, 3775, 3776, 3777, 3778, 3779, 3780, 3781, 3782, and 3783, or substantially similar sequences thereof having at least 90%, at least 95%, at least 98% or at least 99% sequence identity. In some embodiments, the antibody or fragment thereof comprises a HCDR3/LCDR3 amino acid sequence pair comprising SEQ ID NO:3762/3773, 3763/3774, 3764/3775, 3765/3776, 3766/3777, 3767/3778, 3768/3779, 3769/3780, 3770/3781, 3771/3782, or 3772/3783. In some embodiments, the antibody or fragment thereof comprises a HCDR3/LCDR3 amino acid sequence pair comprising SEQ ID NO:3762/3773, 3763/3774, 3764/3775, 3766/3777, 3767/3778, 3769/3780, or 3772/3783. In some embodiments, the antibody or fragment thereof comprises a HCDR3/LCDR3 amino acid sequence pair comprising SEQ ID NO:3766/3777.

In some embodiments, the antibody or fragment thereof further comprises a heavy chain CDR1 (HCDR1) amino acid sequence selected from the group consisting of SEQ ID NO:3784, 3785, 3786, 3787, 3788, 3789, 3790, 3791, 3792, 3793, and 3794, or a substantially similar sequence thereof having at least 90%, at least 95%, at least 98% or at least 99% sequence identity; and a heavy chain CDR2 (HCDR2) amino acid sequence selected from the group consisting of SEQ ID NO:3795, 3796, 3797, 3798, 3799, 3800, 3801, 3802, 3803, 3804, and 3805, or a substantially similar sequence thereof having at least 90%, at least 95%, at least 98% or at least 99% sequence identity; and optionally further comprises a light chain CDR1 (LCDR1) amino acid sequence selected from the group consisting of SEQ ID NO:3806, 3807, 3808, 3809, 3810, 3811, 3812, 3813, 3814, 3815, and 3816, or a substantially similar sequence thereof having at least 90%, at least 95%, at least 98% or at least 99% sequence identity; and/or a light chain CDR2 (LCDR2) amino acid sequence selected from the group consisting of SEQ ID NO:3817 (AAS), 3818 (KAS), 3819 (AAS), 3820 (KAS), 3821 (KAS), 3822 (TTS), 3823 (PAS), 3824 (TAS), 3825 (KVS), 3826 (VAA), and 3827, or a substantially similar sequence thereof having at least 90%, at least 95%, at least 98% or at least 99% sequence identity.

In some embodiments, the antibody or antigen-binding fragment thereof comprises a HCDR1/HCDR2/HCDR3 combination selected from the group consisting of SEQ ID NO:3784/3795/3762, 3785/3796/3763, 3786/3797/3764, 3787/3798/3765, 3788/3799/3766, 3789/3800/3767, 3790/3801/3768, 3791/3802/3769, 3792/3803/3770, 3793/3804/3771, and 3794/3805/3772; and/or a LCDR1/LCDR2/LCDR3 combination selected from the group consisting of SEQ ID NO:3806/3817/3773, 3807/3818/3774, 3808/3819/3775, 3809/3820/3776, 3810/3821/3777, 3811/3822/3778, 3812/3823/3779, 3813/3824/3780, 3814/3825/3781, 3815/3826/3782, and 3816/3827/3783. In some embodiments, the heavy and light chain CDR amino acid sequences comprise a CDR sequence combination selected from the group consisting of SEQ ID NO:3784/3795/3762/3806/3817/3773, 3785/3796/3763/3807/3818/3774, 3786/3797/3764/3808/3819/3775, 3787/3798/3765/3809/3820/3776, 3788/3799/3766/3810/3821/3777, 3789/3800/3767/3811/3822/3778, 3790/3801/3768/3812/3823/3779, 3791/3802/3769/3813/3824/3780, 3792/3803/3770/3814/3825/3781, 3793/3804/3771/3815/3826/3782 and 3794/3805/3772/3816/3827/3783. In some embodiments, the heavy and light chain CDR amino acid sequences comprise a CDR sequence combination of SEQ ID NO: 3784/3795/3762/3806/3817/3773, 3785/3796/3763/3807/3818/3774, 3786/3797/3764/3808/3819/3775, 3788/3799/3766/3810/3821/3777, 3789/3800/3767/3811/3822/3778, 3791/3802/3769/3813/3824/3780 or 3794/3805/3772/3816/3827/3783. In some embodiments, the heavy and light chain CDR amino acid sequences comprise a CDR sequence combination of SEQ ID NO:3788/3799/3766/3810/3821/3777.

In some embodiments, the antibody or antigen-binding fragment thereof, which specifically binds ANGPTL3, comprises heavy and light chain CDR domains contained within HCVR/LCVR pairs selected from the group consisting of SEQ ID NO:3740/3751, 3741/3752, 3742/3753, 3743/3754, 3744/3755, 3745/3756, 3746/3757, 3747/3758, 3748/3759, 3749/3760, and 3750/3761. Methods and techniques for identifying CDRs within HCVR and LCVR amino acid sequences are known in the art and can be applied to identify CDRs within the specified HCVR and/or LCVR amino acid sequences disclosed herein. Conventional definitions that can be applied to identify the boundaries of CDRs include the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol., 1997, 273, 927-948; and Martin et al., Proc. Natl. Acad. Sci. USA, 1989, 86, 9268-9272. Public databases are also available for identifying CDR sequences within an antibody. In some embodiments, the antibody or fragment thereof comprises CDR sequences contained within a HCVR and LCVR pair of SEQ ID NO:3740/3751, 3741/3752, 3742/3753, 3744/3755, 3745/3756, 3747/3758, or 3750/3761. In some embodiments, the antibody or fragment thereof comprises CDR sequences contained within a HCVR and LCVR pair of SEQ ID NO:3744/3755.

In some embodiments, the antibody or antigen-binding fragment thereof competes for specific binding to ANGPTL3 with an antibody or antigen-binding fragment comprising heavy and light chain CDR sequences contained in a HCVR/LCVR sequence pair of SEQ ID NO:3740/3751, 3741/3752, 3742/3753, 3743/3754, 3744/3755, 3745/3756, 3746/3757, 3747/3758, 3748/3759, 3749/3760, or 3750/3761. In some embodiments, the antibody or antigen-binding fragment thereof competes for specific binding to ANGPTL3 with an antibody or fragment thereof comprising a HCVR/LCVR sequence pair of SEQ ID NO:3744/3755. In some embodiments, the antibody or antigen-binding fragment thereof competes for specific binding to ANGPTL3 with an antibody or fragment thereof comprising a heavy and light chain CDR sequence combination selected from the group consisting of 3784/3795/3762/3806/3817/3773, 3785/3796/3763/3807/3818/3774, 3786/3797/3764/3808/3819/3775, 3787/3798/3765/3809/3820/3776, 3788/3799/3766/3810/3821/3777, 3789/3800/3767/3811/3822/3778, 3790/3801/3768/3812/3823/3779, 3791/3802/3769/3813/3824/3780, 3792/3803/3770/3814/3825/3781, 3793/3804/3771/3815/3826/3782 and 3794/3805/3772/3816/3827/3783. In some embodiments, the antibody or antigen-binding fragment thereof competes for specific binding to ANGPTL3 with an antibody or fragment thereof comprising a heavy and light chain CDR sequence combination of SEQ ID NOS:3788/3799/3766/3810/3821/3777.

In some embodiments, the antibody or antigen-binding fragment thereof binds the same epitope on ANGPTL3 that is recognized by an antibody or fragment thereof comprising heavy and light chain CDR sequences from a HCVR/LCVR sequence pair of SEQ ID NO:3740/3751, 3741/3752, 3742/3753, 3743/3754, 3744/3755, 3745/3756, 3746/3757, 3747/3758, 3748/3759, 3749/3760, or 3750/3761. In some embodiments, the antibody or antigen-biding fragment thereof binds the same epitope on ANGPTL3 as that recognized by the antibody or fragment thereof comprising a HCVR/LCVR sequence pair of SEQ ID NO:3744/3755. In some embodiments, the antibody or fragment thereof binds the same epitope on ANGPTL3 that is recognized by an antibody or fragment thereof comprising a heavy and light chain CDR sequence combination selected from the group consisting of 3784/3795/3762/3806/3817/3773, 3785/3796/3763/3807/3818/3774, 3786/3797/3764/3808/3819/3775, 3787/3798/3765/3809/3820/3776, 3788/3799/3766/3810/3821/3777, 3789/3800/3767/3811/3822/3778, 3790/3801/3768/3812/3823/3779, 3791/3802/3769/3813/3824/3780, 3792/3803/3770/3814/3825/3781, 3793/3804/3771/3815/3826/3782 and 3794/3805/3772/3816/3827/3783. In some embodiments, such an epitope is recognized by an antibody or fragment thereof comprising a heavy and light chain CDR sequence combination of SEQ ID NO:3788/3799/3766/3810/3821/3777.

In some embodiments, an isolated anti-ANGPTL3 antibody or antigen-binding fragment thereof that binds to an epitope situated within the N-terminal coiled-coil region at residues 17 to 209 of SEQ ID NO:3739 and neutralizes, inhibits, abrogates, reduces or interferes with, at least one activity of ANGPTL3. In some embodiments, the isolated antibody or antigen-binding fragment thereof specifically binds to an epitope situated within the N-terminal coiled-coil region of ANGPTL3 (SEQ ID NO:3739) and neutralizes, inhibits, abrogates, reduces or interferes with, at least one activity of ANGPTL3, with the proviso that the antibody or fragment thereof does not bind to the ANGPTL3 peptide of SEQ ID NO:3828 (corresponds to residues Glu32 to Leu57 of ANGPTL3 of SEQ ID NO:3739). In some embodiments, the antibody or fragment thereof specifically binds to an epitope within residues 17 to 200, 17 to 100, 17 to 70, 17 to 65, 17 to 60, 17 to 57, or 17 to 50, of ANGPTL3 (SEQ ID NO:3739), optionally with the proviso that the antibody or fragment thereof does not bind to the ANGPTL3 peptide of SEQ ID NO:3828. In some embodiments, the antibody or fragment thereof specifically binds to an epitope within residues 40 to 200, 40 to 100, 40 to 70, 50 to 200, 50 to 100, 50 to 70, 58 to 200, 58 to 100, 58 to 70, 58 to 68, or 61 to 66, of ANGPTL3 (SEQ ID NO:3739), optionally with the proviso that the antibody or fragment thereof does not bind to the ANGPTL3 peptide of SEQ ID NO:3828. In some embodiments, the antibody or antibody fragment binds an epitope which may involve more than one of the enumerated epitopes or residues within the N-terminal coiled-coil region of ANGPTL3, optionally with the proviso that the antibody or fragment thereof does not bind to the ANGPTL3 peptide of SEQ ID NO:3828.

In some embodiments, the antibody or fragment thereof comprises a HCVR encoded by a nucleic acid sequence selected from the group consisting of SEQ ID NO:3829, 3830, 3831, 3832, 3833, 3834, 3835, 3836, 3837, 3838, and 3839, or a substantially identical sequence having at least 90%, at least 95%, at least 98%, or at least 99% homology thereof. In some embodiments, the antibody or fragment thereof comprises a HCVR encoded by a nucleic acid sequence of SEQ ID NO:3829, 3830, 3831, 3833, 3834, 3836, or 3839. In some embodiments, the antibody or fragment thereof comprises a HCVR encoded by a nucleic acid sequence of SEQ ID NO:3833.

In some embodiments, an antibody or antigen-binding fragment thereof comprises a LCVR encoded by a nucleic acid sequence selected from the group consisting of SEQ ID NO:3840, 3841, 3842, 3843, 3844, 3845, 3846, 3847, 3848, 3849, and 3850, or a substantially identical sequence having at least 90%, at least 95%, at least 98%, or at least 99% homology thereof. In some embodiments, the antibody or fragment thereof comprises a LCVR encoded by a nucleic acid sequence of SEQ ID NO:3840, 3841, 3842, 3844, 3845, 3847, or 3850. In some embodiments, the antibody or fragment thereof comprises a LCVR encoded by a nucleic acid sequence of SEQ ID NO:3844.

In some embodiments, the antibody or fragment thereof comprises a HCVR and LCVR (HCVR/LCVR) sequence pair encoded by a nucleic acid sequence pair selected from the group consisting of SEQ ID NO:3829/3840, 3830/3841, 3831/3842, 3832/3843, 3833/3844, 3834/3845, 3835/3846, 3836/3847, 3837/3848, 3838/3849, and 3839/3850. In some embodiments, the antibody or fragment thereof comprises a HCVR/LCVR sequence pair encoded by a nucleic acid sequence pair of SEQ ID NO:3829/3840, 3830/3841, 3831/3842, 3833/3844, 3834/3845, 3836/3847, or 3839/3850. In some embodiments, the antibody or fragment thereof comprises a HCVR/LCVR sequence pair encoded by a nucleic acid sequence pair of SEQ ID NO:3833/3844.

In some embodiments, the antibody or antigen-binding fragment thereof comprises a HCDR3 domain encoded by a nucleotide sequence selected from the group consisting of SEQ ID NO:3851, 3852, 3853, 3854, 3855, 3856, 3857, 3858, 3859, 3860, and 3861, or a substantially identical sequence having at least 90%, at least 95%, at least 98%, or at least 99% homology thereof; and a LCDR3 domain encoded by a nucleotide sequence selected from the group consisting of SEQ ID NO:3862, 3863, 3864, 3865, 3866, 3867, 3868, 3869, 3870, 3871, and 3872, or a substantially identical sequence having at least 90%, at least 95%, at least 98%, or at least 99% homology thereof. In some embodiments, the antibody or fragment thereof comprises a HCDR3 and LCDR3 sequence pair encoded by the nucleic acid sequence pair selected from the group consisting of SEQ ID NO:3851/3862, 3852/3863, 3853/3864, 3854/3865, 3855/3866, 3856/3867, 3857/3868, 3858/3869, 3859/3870, 3860/3871 and 3861/3872. In some embodiments, the antibody or fragment thereof comprises a HCDR3 and LCDR3 sequence pair encoded by the nucleic acid sequence pair of SEQ ID NO:3851/3862, 3852/3863, 3853/3864, 3855/3866, 3856/3867, 3858/3869 or 3861/3872. In some embodiments, the antibody or fragment thereof comprises a HCDR3 and LCDR3 sequence pair encoded by the nucleic acid sequence pair of SEQ ID NO:3855/3866.

In some embodiments, the antibody or fragment thereof further comprises a HCDR1 domain encoded by a nucleotide sequence selected from the group consisting of SEQ ID NO:3873, 3874, 3875, 3876, 3877, 3878, 3879, 3880, 3881, 3882, and 3883, or a substantially identical sequence having at least 90%, at least 95%, at least 98%, or at least 99% homology thereof; and a HCDR2 domain encoded by a nucleotide sequence selected from the group consisting of SEQ ID NO:3884, 3885, 3886, 3887, 3888, 3889, 3890, 3891, 3892, 3893, and 3894, or a substantially identical sequence having at least 90%, at least 95%, at least 98%, or at least 99% homology thereof; and optionally further comprises a LCDR1 domain encoded by a nucleotide sequence selected from the group consisting of SEQ ID NO:3895, 3896, 3897, 3898, 3899, 3900, 3901, 3902, 3903, 3904, and 3905, or a substantially identical sequence having at least 90%, at least 95%, at least 98%, or at least 99% homology thereof; and/or a LCDR2 domain encoded by a nucleotide sequence selected from the group consisting of SEQ ID NO:3906 (gctgcatcc), 3907 (aaggcgtct), 3908 (gctgcatcc), 3909 (aaggcgtct), 3910 (aaggcgtct), 3911 (actacttcc), 3912 (cctgcatcc), 3913 (actgcatcc), 3914 (aaggtttct), 3915 (gttgcagcc), and 3916, or a substantially identical sequence having at least 90%, at least 95%, at least 98%, or at least 99% homology thereof.

In some embodiments, the antibody or antigen-binding fragment thereof comprises a HCDR1/HCDR2/HCDR3 combination encoded by a nucleotide sequence combination selected from the group consisting of SEQ ID NO:3873/3884/3851, 3874/3885/3852, 3875/3886/3853, 3876/3887/3854, 3877/3888/3855, 3878/3889/3856, 3879/3890/3857, 3880/3891/3858, 3881/3892/3859, 3882/3893/3860, and 3883/3894/3861; and/or a LCDR1/LCDR2/LCDR3 combination encoded by a nucleotide sequence combination selected from the group consisting of SEQ ID NO:3895/3906/3862, 3896/3907/3863, 3897/3908/3864, 3898/3909/3865, 3899/3910/3866, 3900/3911/3867, 3901/3912/3868, 3902/3913/3869, 3903/3914/3870, 3904/3915/3871, and 3905/3916/3872. In some embodiments, the antibody or fragment thereof comprises heavy and light chain CDR sequences encoded by a nucleotide sequence combination of SEQ ID NO:3877/3888/3855/3899/3910/3866.

In some embodiments, the anti-ANGPTL3 antibody or antigen-binding fragment thereof comprises a heavy chain variable region (HCVR) encoded by nucleotide sequence segments derived from V_{H}, D_{H} and J_{H} germline sequences, and a light chain variable region (LCVR) encoded by nucleotide sequence segments derived from V_{K} and J_{K} germline sequences, wherein the HCVR and the LCVR are encoded by nucleotide sequence segments derived from a germline gene combination selected from the group consisting of: (i) V_{H}3-43, D_{H}3-3, 43, V_{K}1-5 and J_{K}2; (ii) V_{H}3-11, D_{H}1-1, J_{H}4, V_{K}1-39 and J_{K}4; (iii) V_{H}3-30, D_{H}1-7, J_{H}6, V_{K}1-5 and J_{K}1; (iv) V_{H}3-30, D_{H}1-26, J_{H}6, V_{K}1-12 and J_{K}3; (v) V_{H}3-30, D_{H}3-10, J_{H}6, V_{K}1-12 and J_{K}3; and (vi) V_{H}3-23, D_{H}3-10, J_{H}4, V_{K}1-5 and J_{K}1.

In some embodiments, the antibody or antigen-binding fragment thereof specifically binds to ANGPTL3 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-ANGPTL3 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies, or increase affinities of the antibodies. In yet other applications, modification of galactosylation can be made in order to modify complement dependent cytotoxicity (CDC).

In some embodiments, the ANGPTL3 antibody is evinacumab.

In some embodiments, the SLC5A2 inhibitor comprises an inhibitory nucleic acid molecule. Examples of inhibitory nucleic acid molecules include, but are not limited to, antisense nucleic acid molecules, siRNAs, and shRNAs. Such inhibitory nucleic acid molecules can be designed to target any region of an SLC5A2 nucleic acid molecule. In some embodiments, the antisense RNA, siRNA, or shRNA hybridizes to a sequence within an SLC5A2 genomic nucleic acid molecule or mRNA molecule and decreases expression of the SLC5A2 polypeptide in a cell in the subject. In some embodiments, the SLC5A2 inhibitor comprises an antisense molecule that hybridizes to an SLC5A2 genomic nucleic acid molecule or mRNA molecule and decreases expression of the SLC5A2 polypeptide in a cell in the subject. In some embodiments, the SLC5A2 inhibitor comprises an siRNA that hybridizes to an SLC5A2 genomic nucleic acid molecule or mRNA molecule and decreases expression of the SLC5A2 polypeptide in a cell in the subject. In some embodiments, the SLC5A2 inhibitor comprises an shRNA that hybridizes to an SLC5A2 genomic nucleic acid molecule or mRNA molecule and decreases expression of the SLC5A2 polypeptide in a cell in the subject.

In some embodiments, the SLC5A2 antisense nucleic acid molecules comprise or consist of any of the nucleotide sequences represented by SEQ ID NOs: 1190-1569. In some embodiments, the SLC5A2 siRNA molecules comprise or consist of any of the nucleotide sequences (sense and antisense strands presented one after the other) represented by SEQ ID NOs: 1570-3737 (e.g., the sense strand is, for example, SEQ ID NO:1570 and the corresponding antisense strand is SEQ ID NO:1571; the sense strand is, for example, SEQ ID NO:3736 and the corresponding antisense strand is SEQ ID NO:3737; etc.).

In some embodiments, the SLC5A2 inhibitor comprises a nuclease agent that induces one or more nicks or double-strand breaks at a recognition sequence(s) or a DNA-binding protein that binds to a recognition sequence within an SLC5A2 genomic nucleic acid molecule. The recognition sequence can be located within a coding region of the SLC5A2 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the SLC5A2 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an SLC5A2 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of SLC5A2 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an SLC5A2 genomic nucleic acid molecule or it can be a nickase that creates a single-strand break in an SLC5A2 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be joined or fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of SLC5A2 genomic nucleic acid molecules can be generated by contacting a cell with a Cas protein and one or more gRNAs that hybridize to one or more gRNA recognition sequences within a target genomic locus in the SLC5A2 genomic nucleic acid molecule. The gRNA recognition sequence can include or be proximate to the start codon of an SLC5A2 genomic nucleic acid molecule or the stop codon of an SLC5A2 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an SLC5A2 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an SLC5A2 genomic nucleic acid molecule. An exemplary gRNA is a gRNA effective to direct a Cas enzyme to bind to or cleave an SLC5A2 genomic nucleic acid molecule, wherein the gRNA comprises a DNA-targeting segment that hybridizes to a gRNA recognition sequence within the SLC5A2 genomic nucleic acid molecule. Exemplary gRNAs comprise a DNA-targeting segment that hybridizes to a gRNA recognition sequence present within an SLC5A2 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the target SLC5A2 genomic nucleic acid molecule. The Cas protein can cleave the nucleic acid molecule at a site within or outside of the nucleic acid sequence present in the target SLC5A2 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the SLC5A2 genomic nucleic acid molecule to which a DNA-targeting segment of a gRNA will bind.

Such methods can result, for example, in an SLC5A2 genomic nucleic acid molecule in which a region of the SLC5A2 genomic nucleic acid molecule is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the SLC5A2 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the SLC5A2 inhibitor targets the SGLT2 polypeptide (the polypeptide encoded by the SLC5A2 gene). In some embodiments, the SLC5A2 inhibitor comprises INVOKANA® (canagliflozin), FORXIGA® (dapagliflozin), JARDIANCE® (empagliflozin), SUGLAT® (ipragliflozin), LUSEFI® (luseogliflozin), or APLEWAY® (tofogliflozin), or any combination thereof. In some embodiments, the SLC5A2 inhibitor comprises canagliflozin, dapagliflozin, empagliflozin, ipragliflozin, luseogliflozin, or tofogliflozin, or any combination thereof. In some embodiments, the SLC5A2 inhibitor comprises canagliflozin. In some embodiments, the SLC5A2 inhibitor comprises dapagliflozin. In some embodiments, the SLC5A2 inhibitor comprises empagliflozin. In some embodiments, the SLC5A2 inhibitor comprises ipragliflozin. In some embodiments, the SLC5A2 inhibitor comprises luseogliflozin. In some embodiments, the SLC5A2 inhibitor comprises tofogliflozin.

The inhibitory nucleic acid molecules can comprise RNA, DNA, or both RNA and DNA. The inhibitory nucleic acid molecules can also be joined or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be joined or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that join consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:
Sense: mN*mN*/i2FN/mN/i2FN/mN/i2FN/mN/i2FN/mN/i2FN/mN/i2FN/mN/i2FN/mN/i2FN/*mN*/32FN/
Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

In any of the embodiments described herein for antisense molecules and siRNA molecules, the molecules can comprise 1, 2, or 3 additional nucleotides at the 5′ end, 3′ end, or both the 5′ end and 3′ end. In some embodiments the antisense molecules and siRNA molecules comprise 1, 2, or 3 additional nucleotides at the 5′ end. In some embodiments the antisense molecules and siRNA molecules comprise 1, 2, or 3 additional nucleotides at the 3′ end. In some embodiments the antisense molecules and siRNA molecules comprise 1, 2, or 3 additional nucleotides at both the 5′ end and 3′ end.

In any of the embodiments described herein for antisense molecules and siRNA molecules, the molecules can comprise a substantially identical sequence having at least 80%, at least 85%, at least 90%, at least 95%, at least 98%, or at least 99% homology to the nucleotide sequences disclosed herein. In some embodiments, the antisense molecules and siRNA molecules have at least 80% homology to the nucleotide sequences disclosed herein. In some embodiments, the antisense molecules and siRNA molecules have at least 85% homology to the nucleotide sequences disclosed herein. In some embodiments, the antisense molecules and siRNA molecules have at least 90% homology to the nucleotide sequences disclosed herein. In some embodiments, the antisense molecules and siRNA molecules have at least 95% homology to the nucleotide sequences disclosed herein. In some embodiments, the antisense molecules and siRNA molecules have at least 98% homology to the nucleotide sequences disclosed herein. In some embodiments, the antisense molecules and siRNA molecules have at least 99% homology to the nucleotide sequences disclosed herein.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m²—based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the methods further comprise detecting the presence or absence of an ANGPTL3 variant nucleic acid molecule and detecting the presence or absence of an SLC5A2 variant nucleic acid molecule in a biological sample from the subject. The presence or absence of any of the ANGPTL3 variant nucleic acid molecules and SLC5A2 variant nucleic acid molecules can be detected herein. In some embodiments, the methods further comprises administering a kidney disease therapeutic agent to a subject that is: i) ANGPTL3 reference or heterozygous for the ANGPTL3 variant nucleic acid molecule, and ii) SLC5A2 reference or heterozygous for the SLC5A2 variant nucleic acid molecule.

The present disclosure also provides compositions comprising a combination of any of the ANGPTL3 inhibitors and SLC5A2 inhibitors and a second therapeutic agent. The second therapeutic agent may be one or more of any agent such as (1) 3-hydroxy-3-methylglutaryl-coenzyme A (HMG-CoA) reductase inhibitors, such as cerivastatin, atorvastatin, simvastatin, pitavastatin, rosuvastatin, fluvastatin, lovastatin, pravastatin, and the like; (2) inhibitors of cholesterol uptake and/or bile acid re-absorption; (3) niacin, which increases lipoprotein catabolism; (4) fibrates or amphipathic carboxylic acids, which reduce low-density lipoprotein (LDL) level, improve high-density lipoprotein (HDL) and TG levels, and reduce the number of non-fatal heart attacks; and (5) activators of the LXR transcription factor that plays a role in cholesterol elimination such as 22-hydroxycholesterol, or fixed combinations such as ezetimibe plus simvastatin; a statin with a bile resin (e.g., cholestyramine, colestipol, colesevelam), a fixed combination of niacin plus a statin (e.g., niacin with lovastatin); or with other lipid lowering agents such as omega-3-fatty acid ethyl esters (for example, omacor). Furthermore, the second therapeutic agent can be one or more other inhibitors of ANGPTL3 as well as inhibitors of other molecules, such as ANGPTL4, ANGPTL5, ANGPTL6 and proprotein convertase subtilisin/kexin type 9 (PCSK9), which are involved in lipid metabolism, in particular, cholesterol and/or triglyceride homeostasis. Inhibitors of these molecules include small molecules and antibodies that specifically bind to these molecules and block their activity.

The present disclosure also provides methods of treating a subject with a kidney disease therapeutic agent. In some embodiments, the subject has a kidney disease. In some embodiments, the subject is at risk of developing a kidney disease. The methods comprise administering a kidney disease therapeutic agent to the subject. The methods comprise determining whether the subject has an ANGPTL3 variant nucleic acid molecule and whether the subject has an SLC5A2 variant nucleic acid molecule. The determination can be carried out by obtaining or having obtained a biological sample from the subject, and performing or having performed a sequence analysis on the biological sample to determine if the subject has a genotype comprising the ANGPTL3 variant nucleic acid molecule, and the SLC5A2 variant nucleic acid molecule. The methods comprise administering or continuing to administer the kidney disease therapeutic agent to a subject that is homozygous for both the ANGPTL3 variant nucleic acid molecule and the SLC5A2 variant nucleic acid molecule. The methods comprise administering or continuing to administer the kidney disease therapeutic agent and/or an ANGPTL3 inhibitor and an SLC5A2 inhibitor to a subject that is: i) heterozygous for both the ANGPTL3 variant nucleic acid molecule and the SLC5A2 variant nucleic acid molecule, ii) heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule, or iii) ANGPTL3 reference and SLC5A2 reference. The presence of a genotype having the ANGPTL3 variant nucleic acid molecule and the SLC5A2 variant nucleic acid molecule indicates the subject has a decreased risk of developing a kidney disease.

In some embodiments, the kidney disease is chronic kidney disease. In some embodiments, the kidney disease is diabetic kidney disease. In some embodiments, the kidney disease is a kidney stone. In some embodiments, the kidney disease is chronic glomerulonephritis. In some embodiments, the kidney disease is nephronophthisis. In some embodiments, the kidney disease is chronic interstitial nephritis. In some embodiments, the kidney disease is nephrosclerosis.

For subjects that are genotyped or determined to be: i) either ANGPTL3 reference or heterozygous for an ANGPTL3 variant nucleic acid molecule, and ii) either SLC5A2 reference or heterozygous for an SLC5A2 variant nucleic acid molecule, such subjects can be administered an ANGPTL3 inhibitor and an SLC5A2 inhibitor, as described herein.

In some embodiments, the ANGPTL3 variant nucleic acid molecule is a missense variant, a splice-site variant, a stop-gain variant, a start-loss variant, a stop-loss variant, a frameshift variant, an in-frame indel variant, or a variant that encodes a truncated ANGPTL3 polypeptide. In some embodiments, the SLC5A2 variant nucleic acid molecule is a missense variant, a splice-site variant, a stop-gain variant, a start-loss variant, a stop-loss variant, a frameshift variant, an in-frame indel variant, or a variant that encodes a truncated SLC5A2 polypeptide.

Detecting the presence or absence of an ANGPTL3 variant nucleic acid molecule and an SLC5A2 variant nucleic acid molecule in a biological sample from a subject and/or determining whether a subject has an ANGPTL3 variant nucleic acid molecule an SLC5A2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the subject is ANGPTL3 reference or heterozygous for the ANGPTL3 variant nucleic acid molecule, and SLC5A2 reference or heterozygous for the SLC5A2 variant nucleic acid molecule, and the subject is administered or continued to be administered the kidney disease therapeutic agent and/or an ANGPTL3 inhibitor and an SLC5A2 inhibitor. The ANGPTL3 inhibitor and the SLC5A2 inhibitor can be any of the inhibitors described here, or any combination thereof.

In some embodiments, the treatment or prevention methods comprise detecting the presence or absence of an ANGPTL3 variant polypeptide and an SLC5A2 variant polypeptide in a biological sample from the subject. In some embodiments, when the subject does not have an ANGPTL3 variant polypeptide and an SLC5A2 variant polypeptide, the subject is administered a kidney disease therapeutic agent and is administered an ANGPTL3 inhibitor and an SLC5A2 inhibitor.

The present disclosure also provides methods of treating a subject with a kidney disease therapeutic agent, wherein the subject has a kidney disease or is at risk of developing a kidney disease. In some embodiments, the method comprises determining whether the subject has an ANGPTL3 variant polypeptide and an SLC5A2 variant polypeptide by obtaining or having obtained a biological sample from the subject, and performing or having performed an assay on the biological sample to determine if the subject has an ANGPTL3 variant polypeptide and an SLC5A2 variant polypeptide. When the subject does not have an ANGPTL3 variant polypeptide and an SLC5A2 variant polypeptide, the kidney disease therapeutic agent is administered or continued to be administered to the subject and/or an ANGPTL3 inhibitor and an SLC5A2 inhibitor is administered to the subject. The presence of an ANGPTL3 variant polypeptide and an SLC5A2 variant polypeptide indicates the subject has a decreased risk of developing a kidney disease. In some embodiments, the subject has an ANGPTL3 variant polypeptide and an SLC5A2 variant polypeptide. In some embodiments, the subject does not have an ANGPTL3 variant polypeptide and an SLC5A2 variant polypeptide.

Detecting the presence or absence of an ANGPTL3 variant polypeptide and an SLC5A2 variant polypeptide in a biological sample from a subject and/or determining whether a subject has an ANGPTL3 variant polypeptide and an SLC5A2 variant polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

Examples of kidney disease therapeutic agents that treat chronic kidney disease and/or diabetic kidney disease include, but are not limited to, erythropoietin, a diuretic (such as, for example, furosemide, bumetanide, ethacrynic acid, metolazone, and hydrochlorothiazide), a blood pressure medication, a phosphate binder, sodium bicarbonate, a cholesterol medication, and a gliflozin, or any combination thereof.

Examples of kidney disease therapeutic agents that treat or inhibit a kidney stone include, but are not limited to, potassium citrate, a diuretic (such as, for example, furosemide, bumetanide, ethacrynic acid, metolazone, and hydrochlorothiazide), allopurinol, acetohydroxamic acid, tamsulosin, nifedipine, d-penicillamine, tiopronin, and mercaptopropionyl glycine, or any combination thereof.

Examples of kidney disease therapeutic agents that treat or inhibit chronic glomerulonephritis include, but are not limited to, an angiotensin-converting enzyme (ACE) inhibitor (such as, for example, lisinopril, enalapril, captopril, benazepril, fosinopril, and quinapril), a diuretic (such as, for example, furosemide, bumetanide, ethacrynic acid, metolazone, and hydrochlorothiazide), a calcium channel blocker (such as, for example, amlodipine, nifedipine, felodipine, isradipine, verapamil, and diltiazem), a beta-adrenergic blocker (such as, for example, metoprolol, bisoprolol, esmolol, atenolol, propranolol, sotalol, labetalol, pindolol, and penbutolol), an alpha-adrenergic agonist (such as, for example, clonidine, tizanidine, and dexmedetomidine), a corticosteroid (such as, for example, prednisone), and an immunosuppressant (such as, for example, cyclosphosphamide), or any combination thereof.

Examples of kidney disease therapeutic agents that treat or inhibit nephronophthisis include, but are not limited to, erythropoietin and a blood pressure medication, or any combination thereof.

Examples of kidney disease therapeutic agents that treat or inhibit chronic interstitial nephritis include, but are not limited to, a corticosteroid, erythropoietin, a blood pressure medication, a statin, and a chelating agent (such as, for example, succimer and edetate calcium disodium), or a combination thereof.

Examples of kidney disease therapeutic agents that treat or inhibit nephrosclerosis include, but are not limited to, a diuretic (such as, for example, furosemide, bumetanide, ethacrynic acid, metolazone, and hydrochlorothiazide), an ACE inhibitor (such as, for example, lisinopril, enalapril, captopril, benazepril, fosinopril, and quinapril), an ARB (such as, for example, losartan, and valsartan), a calcium channel blocker (such as, for example, amlodipine, nifedipine, felodipine, isradipine, verapamil, and diltiazem), a beta-adrenergic blocker (such as, for example, metoprolol, bisoprolol, esmolol, atenolol, propranolol, sotalol, labetalol, pindolol, and penbutolol), an alpha-adrenergic agonist (such as, for example, clonidine, tizanidine, and dexmedetomidine), a renin inhibitor (such as, for example, aliskiren), a vasodilator (such as, for example, minoxidil and hydralazine), and an alpha-1 blocker (such as, for example, doxazosin), or any combination thereof.

In some embodiments, the dose of the kidney disease therapeutic agents can be decreased by about 10%, by about 20%, by about 30%, by about 40%, by about 50%, by about 60%, by about 70%, by about 80%, or by about 90% for subjects that are ANGPTL3 reference or heterozygous for an ANGPTL3 variant nucleic acid molecule and that are SLC5A2 reference or heterozygous for an SLC5A2 variant nucleic acid molecule compared to subjects that are homozygous for an ANGPTL3 variant nucleic acid molecule and homozygous for an ANGPTL3 variant nucleic acid molecule. In some embodiments, the dose of the kidney disease therapeutic agents can be decreased by about 10%, by about 20%, by about 30%, by about 40%, or by about 50%. In addition, the subjects that are ANGPTL3 reference or heterozygous for an ANGPTL3 variant nucleic acid molecule and that are SLC5A2 reference or heterozygous for an SLC5A2 variant nucleic acid molecule can be administered less frequently compared to subjects that are homozygous for an ANGPTL3 variant nucleic acid molecule and homozygous for an ANGPTL3 variant nucleic acid molecule.

Administration of the kidney disease therapeutic agents, ANGPTL3 inhibitors, and/or SLC5A2 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the kidney disease therapeutic agents, ANGPTL3 inhibitors, and/or SLC5A2 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in a kidney disease, a decrease/reduction in the severity of a kidney disease (such as, for example, a reduction or inhibition of development of a kidney disease), a decrease/reduction in symptoms and kidney disease-related effects, delaying the onset of symptoms and kidney disease-related effects, reducing the severity of symptoms of kidney disease-related effects, reducing the number of symptoms and kidney disease-related effects, reducing the latency of symptoms and kidney disease-related effects, an amelioration of symptoms and kidney disease-related effects, reducing secondary symptoms, reducing secondary infections, preventing relapse to a kidney disease, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of a kidney disease development/progression (such as, for example, a complete or partial avoidance/inhibition or a delay), and an increased survival time of the affected host animal, following administration of a therapeutic protocol. Treatment of a kidney disease encompasses the treatment of a subject already diagnosed as having any form of a kidney disease at any clinical stage or manifestation, the delay of the onset or evolution or aggravation or deterioration of the symptoms or signs of a kidney disease, and/or preventing and/or reducing the severity of a kidney disease.

The present disclosure also provides methods of identifying a subject having an increased risk of developing a kidney disease. The methods comprise determining or having determined the presence or absence of an ANGPTL3 variant nucleic acid molecule and determining or having determined the presence or absence of an SLC5A2 variant nucleic acid molecule in a biological sample obtained from the subject. When the subject is ANGPTL3 reference and SLC5A2 reference, then the subject has an increased risk of developing a kidney disease. When the subject is heterozygous or homozygous for the ANGPTL3 variant nucleic acid molecule and heterozygous or homozygous for the SLC5A2 variant nucleic acid molecule, or when the subject is heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule, then the subject has a decreased risk of developing a kidney disease.

In some embodiments, the kidney disease is chronic kidney disease. In some embodiments, the kidney disease is diabetic kidney disease. In some embodiments, the kidney disease is a kidney stone. In some embodiments, the kidney disease is chronic glomerulonephritis. In some embodiments, the kidney disease is nephronophthisis. In some embodiments, the kidney disease is chronic interstitial nephritis. In some embodiments, the kidney disease is nephrosclerosis.

In some embodiments, the ANGPTL3 variant nucleic acid molecule is a missense variant, a splice-site variant, a stop-gain variant, a start-loss variant, a stop-loss variant, a frameshift variant, an in-frame indel variant, or a variant that encodes a truncated ANGPTL3 polypeptide. In some embodiments, the ANGPTL3 variant nucleic acid molecule is any of the ANGPTL3 variant nucleic acid molecules described herein.

In some embodiment, the SLC5A2 variant nucleic acid molecule is a missense variant, a splice-site variant, a stop-gain variant, a start-loss variant, a stop-loss variant, a frameshift variant, an in-frame indel variant, or a variant that encodes a truncated SLC5A2 polypeptide. In some embodiments, the SLC5A2 variant nucleic acid molecule is any of the SLC5A2 variant nucleic acid molecules described herein.

Having a single copy of an ANGPTL3 variant nucleic acid molecule and a single copy of an SLC5A2 variant nucleic acid molecule is more protective of a subject from developing a kidney disease than having no copies of an ANGPTL3 variant nucleic acid molecule and no copies of an SLC5A2 variant nucleic acid molecule. Without intending to be limited to any particular theory or mechanism of action, it is believed that a single copy of an ANGPTL3 variant nucleic acid molecule and a single copy of an SLC5A2 variant nucleic acid molecule an SLC5A2 variant nucleic acid molecule (i.e., heterozygous for an ANGPTL3 variant nucleic acid molecule and an SLC5A2 variant nucleic acid molecule, or heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule) is protective of a subject from developing a kidney disease, and it is also believed that having two copies of an ANGPTL3 variant nucleic acid molecule and two copies of an SLC5A2 variant nucleic acid molecule (i.e., homozygous for an ANGPTL3 variant nucleic acid molecule and an SLC5A2 variant nucleic acid molecule) may be more protective of a subject from developing a kidney disease, relative to a subject with a single copy of each. Thus, in some embodiments, a single copy of an ANGPTL3 variant nucleic acid molecule and a single copy of an SLC5A2 variant nucleic acid molecule may not be completely protective, but instead, may be partially or incompletely protective of a subject from developing a kidney disease. While not desiring to be bound by any particular theory, there may be additional factors or molecules involved in the development of a kidney disease that are still present in a subject having a single copy of an ANGPTL3 variant nucleic acid molecule and a single copy of an SLC5A2 variant nucleic acid molecule, thus resulting in less than complete protection from the development of a kidney disease.

Determining whether a subject has an ANGPTL3 variant nucleic acid molecule and an SLC5A2 variant nucleic acid molecule in a biological sample from a subject and/or determining whether a subject has an ANGPTL3 variant nucleic acid molecule an SLC5A2 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, the methods further comprising administering a kidney disease therapeutic agent and/or an ANGPTL3 inhibitor and an SLC5A2 inhibitor to a subject that is: i) ANGPTL3 reference or heterozygous for the ANGPTL3 variant nucleic acid molecule, and ii) SLC5A2 reference or heterozygous for the SLC5A2 variant nucleic acid molecule. In some embodiments, the ANGPTL3 inhibitor and the SLC5A2 inhibitor comprises any of the inhibitors described herein. In some embodiments, the ANGPTL3 inhibitor comprises an inhibitory nucleic acid molecule that hybridizes to an ANGPTL3 nucleic acid molecule. In some embodiments, the inhibitory nucleic acid molecule comprises an antisense nucleic acid molecule, an siRNA, or an shRNA. In some embodiments, the inhibitory nucleic acid molecule comprises an antisense nucleic acid molecule. In some embodiments, the inhibitory nucleic acid molecule comprises an siRNA. In some embodiments, the ANGPTL3 inhibitor comprises a small molecule. In some embodiments, the ANGPTL3 inhibitor comprises an antibody. In some embodiments, the antibody comprises evinacumab. In some embodiments, the SLC5A2 inhibitor comprises an inhibitory nucleic acid molecule that hybridizes to a SLC5A2 nucleic acid molecule. In some embodiments, the inhibitory nucleic acid molecule comprises an antisense nucleic acid molecule, an siRNA, or an shRNA. In some embodiments, the inhibitory nucleic acid molecule comprises an antisense nucleic acid molecule. In some embodiments, the inhibitory nucleic acid molecule comprises an siRNA. In some embodiments, the SLC5A2 inhibitor comprises canagliflozin, dapagliflozin, empagliflozin, ipragliflozin, luseogliflozin, or tofogliflozin, or any combination thereof.

The present disclosure also provides methods of determining a subject's aggregate burden, or polygenic risk score (PRS), of having two or more ANGPTL3 and SLC5A2 variant nucleic acid molecules, and/or two or more ANGPTL3 and SLC5A2 variant polypeptides associated with a decreased risk of developing a kidney disease. The aggregate burden is the sum of two or more genetic variants that can be carried out in an association analysis with a kidney disease. In some embodiments, the subject is homozygous for one or more ANGPTL3 and SLC5A2 variant nucleic acid molecules associated with a decreased risk of developing a kidney disease. In some embodiments, the subject is heterozygous for one or more ANGPTL3 and SLC5A2 variant nucleic acid molecules associated with a decreased risk of developing a kidney disease. In some embodiments, the subject is heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule. When the subject has a lower aggregate burden, the subject has an increased risk of developing a kidney disease, and the subject is administered or continued to be administered the kidney disease therapeutic agent and/or an ANGPTL3 inhibitor and SLC5A2 inhibitor. When the subject has a higher aggregate burden, the subject has a decreased risk of developing a kidney disease and the subject is administered or continued to be administered the kidney disease therapeutic agent. The higher the aggregate burden, the lower the risk of developing a kidney disease.

In some embodiments, a subject's aggregate burden of having any two or more ANGPTL3 and SLC5A2 variant nucleic acid molecules represents a weighted sum of a plurality of any of the ANGPTL3 and SLC5A2 variant nucleic acid molecules. In some embodiments, the aggregate burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the ANGPTL3 gene and SLC5A2 gene, where the genetic burden is the number of alleles multiplied by the association estimate with a kidney disease or related outcome for each allele (e.g., a weighted polygenic burden score). In some embodiments, when the subject has an aggregate burden higher than a desired threshold score, the subject has a decreased risk of developing a kidney disease. In some embodiments, when the subject has an aggregate burden lower than a desired threshold score, the subject has an increased risk of developing a kidney disease.

In some embodiments, the aggregate burden may be divided into quintiles, e.g., top quintile, second quintile, intermediate quintile, fourth quintile, and bottom quintile, wherein the top quintile of aggregate burden corresponds to the lowest risk group and the bottom quintile of aggregate burden corresponds to the highest risk group. In some embodiments, a subject having a higher aggregate burden comprises the highest weighted aggregate burdens, including, but not limited to the top 10%, top 20%, top 30%, top 40%, or top 50% of aggregate burdens from a subject population. In some embodiments, the genetic variants comprise the genetic variants having association with a kidney disease in the top 10%, top 20%, top 30%, top 40%, or top 50% of p-value range for the association. In some embodiments, each of the identified genetic variants comprise the genetic variants having association with a kidney disease with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, about or 10⁻¹⁵. In some embodiments, the identified genetic variants comprise the genetic variants having association with a kidney disease with p-value of less than 5×10⁻⁸. In some embodiments, the identified genetic variants comprise genetic variants having association with a kidney disease in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects have aggregate burdens in the bottom decile, quintile, or tertile in a reference population. The threshold of the aggregate burden can be determined on the basis of the nature of the intended practical application and the risk difference that would be considered meaningful for that practical application.

The present disclosure also provides methods of detecting the presence or absence of ANGPTL3 and SLC5A2 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any ANGPTL3 and SLC5A2 variant nucleic acid molecule, preliminary processing designed to isolate or enrich the biological sample for the genomic DNA can be employed. A variety of techniques may be used for this purpose. When detecting the level of any ANGPTL3 and SLC5A2 variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting an ANGPTL3 and an SLC5A2 variant nucleic acid molecule in a subject comprises performing a sequence analysis on a biological sample obtained from the subject to determine whether an ANGPTL3 and an SLC5A2 genomic nucleic acid molecule in the biological sample, and/or an ANGPTL3 and an SLC5A2 mRNA molecule in the biological sample, and/or an ANGPTL3 and an SLC5A2 cDNA molecule produced from an mRNA molecule in the biological sample, is present in the sample. In some embodiments, the methods detect the ANGPTL3 and SLC5A2 variant genomic nucleic acid molecule that comprises any of the the genetic variants described herein, or an mRNA molecule produced therefrom, or a cDNA molecule produced from the mRNA molecule.

In some embodiments, the methods of detecting the presence or absence of an ANGPTL3 and an SLC5A2 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising an ANGPTL3 and an SLC5A2 genomic nucleic acid molecule or mRNA molecule, and if mRNA, optionally reverse transcribing the mRNA into cDNA. Such assays can comprise, for example determining the identity of these positions of the particular ANGPTL3 and SLC5A2 nucleic acid molecules. In some embodiments, the method is an in vitro method.

In some embodiments, the determining step, detecting step, or sequence analysis comprises sequencing at least a portion of the nucleotide sequence of the ANGPTL3 and SLC5A2 genomic nucleic acid molecule, the ANGPTL3 and SLC5A2 mRNA molecule, or the ANGPTL3 and SLC5A2 cDNA molecule in the biological sample that comprises a genetic variation compared to the corresponding ANGPTL3 and SLC5A2 reference molecule. In some embodiments, the sequenced portion comprises one or more variations that cause a loss-of-function (partial or complete) or are predicted to cause a loss-of-function (partial or complete).

In some embodiments, the assay comprises sequencing the entire nucleic acid molecule. In some embodiments, only an ANGPTL3 and an SLC5A2 genomic nucleic acid molecule is analyzed. In some embodiments, only an ANGPTL3 and an SLC5A2 mRNA is analyzed. In some embodiments, only an ANGPTL3 and an SLC5A2 cDNA obtained from the ANGPTL3 and SLC5A2 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to an ANGPTL3 and an SLC5A2 variant genomic sequence, variant mRNA sequence, or variant cDNA sequence and not the corresponding ANGPTL3 and SLC5A2 reference sequence under stringent conditions and determining whether hybridization has occurred.

In some embodiments, the determining step, detecting step, or sequence analysis comprises: a) amplifying at least a portion of the ANGPTL3 and SLC5A2 nucleic acid molecule that encodes the ANGPTL3 and SLC5A2 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ANGPTL3 and an SLC5A2 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to ANGPTL3 and SLC5A2 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ANGPTL3 and SLC5A2 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ANGPTL3 and SLC5A2 variant nucleic acid molecules disclosed herein. The primers described herein can be used to amplify any ANGPTL3 and SLC5A2 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding an ANGPTL3 and an SLC5A2 reference genomic nucleic acid molecule, an ANGPTL3 and an SLC5A2 reference mRNA molecule, and/or an ANGPTL3 and an SLC5A2 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be joined or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be joined or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides kidney disease therapeutic agents for use in the treatment or prevention of a kidney disease in a subject having an ANGPTL3 and an SLC5A2 variant nucleic acid molecule. Any of the kidney disease therapeutic agents described herein can be used herein. Any of the ANGPTL3 and SLC5A2 variant nucleic acid molecules disclosed herein can be used herein. In some embodiments, the ANGPTL3 and SLC5A2 variant nucleic acid molecule is a ANGPTL3 and SLC5A2 variant genomic nucleic acid molecule that comprises any of the genetic variants described herein, or is an mRNA molecule produced therefrom, or is a cDNA molecule produced from the mRNA molecule.

The present disclosure also provides uses of kidney disease therapeutic agents for use in the preparation of a medicament for treating or preventing a kidney disease in a subject having an ANGPTL3 and an SLC5A2 variant nucleic acid molecule. Any of the kidney disease therapeutic agents described herein can be used herein. Any of the ANGPTL3 and SLC5A2 variant nucleic acid molecules disclosed herein can be used herein. In some embodiments, the ANGPTL3 and SLC5A2 variant nucleic acid molecule is a ANGPTL3 and SLC5A2 variant genomic nucleic acid molecule that comprises any of the genetic variants described herein, or is an mRNA molecule produced therefrom, or is a cDNA molecule produced from the mRNA molecule.

The present disclosure also provides ANGPTL3 and SLC5A2 inhibitors for use in the treatment or prevention of a kidney disease in a subject that is ANGPTL3 and SLC5A2 reference or is heterozygous for an ANGPTL3 and an SLC5A2 variant nucleic acid molecule or is heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule. Any of the ANGPTL3 and SLC5A2 inhibitors described herein can be used herein. Any of the ANGPTL3 and SLC5A2 variant nucleic acid molecules disclosed herein can be used herein. In some embodiments, the ANGPTL3 and SLC5A2 variant nucleic acid molecule is an ANGPTL3 and an SLC5A2 variant genomic nucleic acid molecule that comprises any of the genetic variants described herein, or is an mRNA molecule produced therefrom, or is a cDNA molecule produced from the mRNA molecule.

The present disclosure also provides ANGPTL3 and SLC5A2 inhibitors in the preparation of a medicament for treating or preventing a kidney disease in a subject that is ANGPTL3 and SLC5A2 reference or is heterozygous for an ANGPTL3 and an SLC5A2 variant nucleic acid molecule or heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule. Any of the ANGPTL3 and SLC5A2 inhibitors described herein can be used herein. Any of the ANGPTL3 and SLC5A2 variant nucleic acid molecules disclosed herein can be used herein. In some embodiments, the ANGPTL3 and SLC5A2 variant nucleic acid molecule is an ANGPTL3 and an SLC5A2 variant genomic nucleic acid molecule that comprises any of the genetic variants described herein, or is an mRNA molecule produced therefrom, or is a cDNA molecule produced from the mRNA molecule.

In some embodiments, the ANGPTL3 and SLC5A2 inhibitors and the kidney disease therapeutic agent are disposed within a pharmaceutical composition. In some embodiments, the ANGPTL3 and SLC5A2 inhibitors are disposed within a first pharmaceutical composition and the kidney disease therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition. Alternately, the ANGPTL3 inhibitor, the SLC5A2 inhibitor, and the kidney disease therapeutic agent are disposed in separate pharmaceutical compositions.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Example 1: Loss-of-Function of ANGPTL3 is Associated with Improved Kidney Function Independently of SLC5A2

To determine if ANGPTL3 effects on chronic kidney disease are affected by SGLT2 function, exome sequencing was performed in up to 677,048 participants across five studies to identify rare coding mutations. We estimated associations for eGFR with the burden of identified rare loss-of-function and missense variants in ANGPTL3 with and without adjustment for the burden of rare loss-of-function and missense variants in SLC5A2, the gene encoding SGLT2. eGFR is a widely used biomarker of kidney function in which higher levels indicate better function.

In the analysis, the burden of rare (alternative allele frequency (AAF)<0.1%) predicted loss-of-function (pLOF) or missense genetic variants in ANGPTL3 was strongly associated with increased eGFR (Table 1). Importantly, the burden of pLOF or missense genetic variants in ANGPTL3 had a similar effect on increased eGFR after adjustment for rare loss-of-function and missense variants in SLC5A2 (AAF<0.1%), indicating that the two genes act independently of each other to increase eGFR.

TABLE 1
Associations with increased eGFR levels for the
burden of rare (AAF < 0.1%) pLOF or
missense variants in the ANGPTL3 gene (outcome = eGFR)

| | | Effect per | | | |
|---|---|---|---|---|---|
| Genetic | SLC5A2 | allele | | Allele counts | |
| exposure | adjustment | (95% CI) | Pval | (Ref\|Het\|Hom) | AAF |
| ANGPTL3 | Unadjusted | 0.04 (0.01, | 0.0031 | 651,141\| | 0.00226 |
| pLOF | | 0.07) SD | | 2,950\|5 | |
| or 5/5 | | 0.72 (0.24, | | | |
| missense | | 1.20) | | | |
| variants | | mL/min | | | |
| ANGPTL3 | SLC5A2 pLOF | 0.04 (0.02, | 0.0018 | 651,141\| | 0.00226 |
| pLOF | Adjusted for | 0.07) SD | | 2,950\|5 | |
| or 5/5 | or 5/5 | 0.77 (0.29, | | | |
| missense | missense | 1.24) | | | |
| variants | variant carrier | mL/min | | | |
| | status | | | | |

Abbreviations: CI, confidence interval; SD, standard deviation; mL/min, milliliters per minute; AAF, alternative allele frequency; Ref, homozygous reference genotype; Het, heterozygous carrier of rare pLOF or 5/5 missense variant in ANGPTL3; Hom, homozygous carrier of rare pLOF or 5/5 missense variant in ANGPTL3; pLOF, predicted loss of function; 5/5 missense, missense variant predicted to be deleterious by 5 out of 5 in silico prediction algorithms; eGFR, estimated glomerular filtration rate.

Similar results with increased were observed comparing the association for rare ANGPTL3 pLOF variants alone (AAF<1%, excluding missense variants) with and without adjustment for SLC5A2 pLOF variants (see, Table 2), indicating that the association for rare pLOF plus missense variants reflects a loss-of-function in ANGPTL3 that is independent of the effects of loss-of-function in SLC5A2.

TABLE 2
Associations with increased eGFR levels for the burden of rare
(AAF < 1%) pLOF variants in the ANGPTL3 gene (outcome = eGFR)

| | | Effect per | | Genotype | |
|---|---|---|---|---|---|
| Genetic | SLC5A2 | allele | | counts, | |
| exposure | adjustment | (95% CI) | Pval | Ref\|Het\|Hom | AAF |
| ANGPTL3 | Unadjusted | 0.05 (0.01, | 0.014 | 675,519\| | 0.00113 |
| pLOF | | 0.09) SD | | 1,528\|1 | |
| variants | | 0.86 (0.18, | | | |
| | | 1.54) mL/min | | | |
| ANGPTL3 | Adjusted for | 0.05 (0.01, | 0.010 | 675,519\| | 0.00113 |
| pLOF | SLC5A2 | 0.09) SD | | 1,528\|1 | |
| variants | pLOF variant | 0.89 (0.21, | | | |
| | carrier status | 1.57) mL/min | | | |

Abbreviations: CI, confidence interval; SD, standard deviation; mL/min, milliliters per minute; AAF, alternative allele frequency; Ref, homozygous reference genotype; Het, heterozygous carrier of rare pLOF variant in ANGPTL3; Hom, homozygous carrier of rare pLOF variant in ANGPTL3; pLOF, predicted loss of function; eGFR, estimated glomerular filtration rate.

### Example 2: Loss-of-Function of SLC5A2 is Associated with Improved Kidney Function Independently of ANGPTL3

We next compared the association for rare coding variants in SLC5A2 with increased eGFR, with and without adjustment for rare coding variants in ANGPTL3. The burden of pLOF or missense genetic variants in SLC5A2 (AAF<0.1%) had a similar effect on increased eGFR after adjustment for rare loss-of-function and missense variants in ANGPTL3 (AAF<0.1%) compared to without adjustment, indicating that the two genes act independently of each other to increase eGFR (see, Table 3).

TABLE 3
Associations with increased eGFR levels for the
burden of rare (AAF < 0.1%) pLOF or
missense variants in the SLC5A2 gene (outcome = eGFR)

| | | Effect | | Allele counts | |
|---|---|---|---|---|---|
| Genetic | ANGPTL3 | per-allele | | (Ref\|Het\| | |
| Exposure | adjustment | (95% CI) | P | Hom) | AAF |
| SLC5A2 | Unadjusted | 0.06 (0.03, | 1.8E−5 | 650,540\| | 0.0027 |
| pLOF | | 0.08) SD | | 3,551\|5 | |
| or 5/5 | | 0.95 (0.52, | | | |
| missense | | 1.39) mL/min | | | |
| variants | | | | | |
| SLC5A2 | Adjusted for | 0.06 (0.03, | 1.6E−5 | 650,540\| | 0.0027 |
| pLOF | ANGPTL3 | 0.08) SD | | 3,551\|5 | |
| or 5/5 | pLOF or 5/5 | 0.96 (0.53, | | | |
| missense | missense | 1.40) mL/min | | | |
| variants | variant | | | | |
| | carrier | | | | |
| | status | | | | |

Abbreviations: CI, confidence interval; SD, standard deviation; mL/min, milliliters per minute; AAF, alternative allele frequency; Ref, homozygous reference genotype; Het, heterozygous carrier of rare pLOF or 5/5 missense variant in SLC5A2; Hom, homozygous carrier of rare pLOF or 5/5 missense variant in SLC5A2; pLOF, predicted loss of function; 5/5 missense, missense variant predicted to be deleterious by 5 out of 5 in silico prediction algorithms; eGFR, estimated glomerular filtration rate.

Similar results with increased were observed comparing the association for rare SLC5A2 pLOF variants alone (AAF<1%, excluding missense variants) with and without adjustment for ANGPTL3 pLOF variants (see, Table 4), indicating that the association for rare pLOF plus missense variants reflects a loss-of-function in SLC5A2 that is independent of the effects of loss-of-function in ANGPTL3.

TABLE 4
Associations with increased eGFR levels for the burden of rare
(AAF < 1%) pLOF variants in the SLC5A2 gene (outcome = eGFR)

| Genetic | | Effect per-allele | | | |
|---|---|---|---|---|---|
| exposure | Analysis | (95% CI) | P | Allele counts | AAF |
| SLC5A2 | Unadjusted | 0.07 (0.02, | 0.012 | 676,295\|753\|0 | 0.00056 |
| pLOF | | 0.12) SD | | | |
| variants | | 1.24 (0.27, 2.20) | | | |
| | | mL/min | | | |
| SLC5A2 | Adjusted for | 0.07 (0.02, | 0.010 | 676,295\|753\|0 | 0.00056 |
| pLOF | ANGPTL3 | 0.13) SD | | | |
| variants | pLOF variant | 1.27 (0.30, 2.23) | | | |
| | carrier status | mL/min | | | |

Abbreviations: CI, confidence interval; SD, standard deviation; mL/min, milliliters per minute; AAF, alternative allele frequency; Ref, homozygous reference genotype; Het, heterozygous carrier of rare pLOF variant in SLC5A2; Hom, homozygous carrier of rare pLOF variant in SLC5A2; pLOF, predicted loss of function; eGFR, estimated glomerular filtration rate.

### Example 3: Rare Coding Variants in ANGPTL3 and SLC5A2 are Associated with Improved Kidney Function Independently of the Other Gene

To formally test whether the effects of ANGPTL3 and SLC5A2 rare coding variants on eGFR were independent of each other, we performed an interaction analysis. The main effects of ANGPTL3 (that is, the effect of ANGPTL3 variants in individuals with homozygous reference genotype for SLC5A2 variants) and SLC5A2 (that is, the effect of SLC5A2 variants in individuals with homozygous reference genotype for ANGPTL3 variants) on eGFR were similar (see, Table 5) to the overall effects on eGFR of ANGPTL3 rare coding variants (see, Table 1) or SLC5A2 rare coding variants (see, Table 3), respectively. The interaction term, which tests whether the effect of ANGPTL3 variants differs in SLC5A2 homozygous reference vs SLC5A2 heterozygous variants carriers, and whether the effect of SLC5A2 variants differs in ANGPTL3 homozygous reference vs ANGPTL3 heterozygous variants carriers, was not significant (see, Table 5). This indicates that the effects of ANGPTL3 do not depend on SLC5A2 genotype status and that the effects of SLC5A2 do not depend on ANGPTL3 genotype status, and thus that there is no evidence of deviation from additivity for the associations of these two genotypes.

TABLE 5
Interaction between the burdens of rare (AAF < 0.1%)
pLOF or missense variants in the ANGPTL3 and SLC5A2
genes in effects on eGFR (outcome = eGFR)

| | | Effect per-allele | | Allele counts |
|---|---|---|---|---|
| Row | Genetic exposure | (95% CI) | P | (Ref\|Het\|Hom) |
| 1 | ANGPTL3 pLOF or | 0.04 (0.01, | 0.0044 | 629,281\| |
| | 5/5 missense variants | 0.07) SD | | 2,759\|2 |
| | (main effect) | 0.72 (0.23, 1.22) | | |
| | | mL/min | | |
| 2 | SLC5A2 pLOF or 5/5 | 0.06 (0.03, | 1.7E−05 | 629,281\| |
| | missense variants | 0.08) SD | | 3,448\|4 |
| | (main effect) | 0.98 (0.53, 1.42) | | |
| | | mL/min | | |
| 3 | ANGPTL3 pLOF or | −0.18 (−0.52, | 0.31 | 6,207\|15\|1 |
| | 5/5 missense variants | 0.16) SD | | |
| | X | −3.10 (−9.02, | | |
| | SLC5A2 pLOF or 5/5 | 2.83) | | |
| | missense | mL/min | | |
| | variants | | | |
| | (interaction effect) | | | |

Abbreviations: CI, confidence interval; SD, standard deviation; mL/min, milliliters per minute; AAF, alternative allele frequency; Ref, homozygous reference genotype; Het, heterozygous carrier of rare pLOF or 5/5 missense variant in ANGPTL3 (Row 1) or SLC5A2 (Row 2); Hom, homozygous carrier of rare pLOF or 5/5 missense variant in ANGPTL3 (Row 1) or SLC5A2 (Row 2); pLOF, predicted loss of function; 5/5 missense, missense variant predicted to be deleterious by 5 out of 5 in silico prediction algorithms; eGFR, estimated glomerular filtration rate.

The associations between ANGPTL3, SLC5A2, and eGFR were driven by multiple rare pLOF or missense variants in the ANGPTL3 and SLC5A2 genes (see, Table 6).

TABLE 6
Missense or pLOF variants in ANGPTL3 or SLC5A2 that were identified by exome sequencing and included in the gene burden association analyses

| Genomic | | | | | | | |
|---|---|---|---|---|---|---|---|
| coordinate | | | | | | | |
| for the | | | | ANGPTL3 | | SLC5A2 | |
| genetic | Predicted | Coding | Protein | pLOF or | ANGPTL3 | pLOF or | SLC5A2 |
| variant, | consequence | DNA | amino acid | 5/5 ms | pLOF | 5/5 ms | pLOF |
| C:P:R:A | of variant | change | change | variant | variant | variant | variant |
| 1:62597562:T:TA | frameshift | c.1dupA | p.Met1fs | Yes | Yes | No | No |
| 1:62597568:T:G | start lost | c.2T > G | p.Met1? | Yes | Yes | No | No |
| 1:62597608:T:TA | frameshift | c.43dupA | p.Ile15fs | Yes | Yes | No | No |
| 1:62597633:A:AG | frameshift | c.67_68insG | p.Asn23fs | Yes | Yes | No | No |
| 1:62597634:A:AT | frameshift | c.70dupT | p.Ser24fs | Yes | Yes | No | No |
| 1:62597670:C:G | stop gained | c.104C > G | p.Ser35* | Yes | Yes | No | No |
| 1:62597673:G:C | missense (5/5) | c.107G > C | p.Arg36Thr | Yes | No | No | No |
| 1:62597676:T:C | missense (5/5) | c.110T > C | p.Phe37Ser | Yes | No | No | No |
| 1:62597676:T:G | missense (5/5) | c.110T > G | p.Phe37Cys | Yes | No | No | No |
| 1:62597679:C:A | missense (5/5) | c.113C > A | p.Ala38Asp | Yes | No | No | No |
| 1:62597690:G:A | missense (5/5) | c.124G > A | p.Asp42Asn | Yes | No | No | No |
| 1:62597693:G:A | missense (5/5) | c.127G > A | p.Val43Ile | Yes | No | No | No |
| 1:62597694:T:C | missense (5/5) | c.128T > C | p.Val43Ala | Yes | No | No | No |
| 1:62597723:T:G | missense (5/5) | c.157T > G | p.Leu53Val | Yes | No | No | No |
| 1:62597727:G:A | missense (5/5) | c.161G > A | p.Gly54Glu | Yes | No | No | No |
| 1:62597731:TG:T | frameshift | c.167delG | p.Gly56fs | Yes | Yes | No | No |
| 1:62597742:AC:A | frameshift | c.177delC | p.Phe60fs | Yes | Yes | No | No |
| 1:62597743:CTT:C | frameshift | c.179_180delTT | p.Phe60fs | Yes | Yes | No | No |
| 1:62597751:A:G | missense (5/5) | c.185A > G | p.His62Arg | Yes | No | No | No |
| 1:62597754:A:C | missense (5/5) | c.188A > C | p.Lys63Thr | Yes | No | No | No |
| 1:62597757:C:T | missense (5/5) | c.191C > T | p.Thr64Met | Yes | No | No | No |
| 1:62597759:A:T | stop gained | c.193A > T | p.Lys65* | Yes | Yes | No | No |
| 1:62597774:G:A | missense (5/5) | c.208G > A | p.Asp70Asn | Yes | No | No | No |
| 1:62597787:A:C | missense (5/5) | c.221A > C | p.Lys74Thr | Yes | No | No | No |
| 1:62597789:C:T | missense (5/5) | c.223C > T | p.Leu75Phe | Yes | No | No | No |
| 1:62597815:T:G | stop gained | c.249T > G | p.Tyr83* | Yes | Yes | No | No |
| 1:62597823:C:G | missense (5/5) | c.257C > G | p.Ser86Trp | Yes | No | No | No |
| 1:62597846:G:T | stop gained | c.280G > T | p.Glu94* | Yes | Yes | No | No |
| 1:62597855:A:T | stop gained | c.289A > T | p.Lys97* | Yes | Yes | No | No |
| 1:62597896:T:G | missense (5/5) | c.330T > G | p.Asn110Lys | Yes | No | No | No |
| 1:62597901:A:C | missense (5/5) | c.335A > C | p.Glu112Ala | Yes | No | No | No |
| 1:62597909:AAT:A | frameshift | c.346_347delAT | p.Met116fS | Yes | Yes | No | No |
| 1:62597921:GAACTC:G | frameshift | c.363_367delCTCAA | p.Asn121fs | Yes | Yes | No | No |
| 1:62597946:T:C | missense (5/5) | c.380T > C | p.Leu127Pro | Yes | No | No | No |
| 1:62597951:G:T | stop gained | c.385G > T | p.Glu129* | Yes | Yes | No | No |
| 1:62597954:G:GA | frameshift | c.394dupA | p.Ile132fs | Yes | Yes | No | No |
| 1:62597967:T:A | missense (5/5) | c.401T > A | p.Leu134His | Yes | No | No | No |
| 1:62597972:CAA:C | frameshift | c.410_411delAA | p.Lys137fs | Yes | Yes | No | No |
| 1:62597996:CAACT:C | frameshift | c.439_442delAACT | p.Asn147fs | Yes | Yes | No | No |
| 1:62598020:C:T | stop gained | c.454C > T | p.Gln152* | Yes | Yes | No | No |
| 1:62598048:TA:T | frameshift | c.484delA | p.Thr162fs | Yes | Yes | No | No |
| 1:62598059:A:G | missense (5/5) | c.493A > G | p.Lys165Glu | Yes | No | No | No |
| 1:62598059:AAAGT:A | frameshift | c.494_495 + 2delAAGT | p.Lys165fs | Yes | Yes | No | No |
| 1:62598711:C:T | stop gained | c.511C > T | p.Gln171* | Yes | Yes | No | No |
| 1:62598714:G:T | missense (5/5) | c.514G > T | p.Asp172Tyr | Yes | No | No | No |
| 1:62598714:G:C | missense (5/5) | c.514G > C | p.Asp172His | Yes | No | No | No |
| 1:62598715:A:G | missense (5/5) | c.515A > G | p.Asp172Gly | Yes | No | No | No |
| 1:62598724:T:C | missense (5/5) | c.524T > C | p.Ile175Thr | Yes | No | No | No |
| 1:62598753:CA:C | frameshift | c.555delA | p.Gln185fs | Yes | Yes | No | No |
| 1:62598758:T:A | stop gained | c.558T > A | p.Tyr186* | Yes | Yes | No | No |
| 1:62598762:CAATT:C | frameshift | c.565_568delTTAA | p.Leu189fs | Yes | Yes | No | No |
| 1:62598771:CA:C | frameshift | c.573delA | p.Gln191fs | Yes | Yes | No | No |
| 1:62598774:C:T | stop gained | c.574C > T | p.Gln192* | Yes | Yes | No | No |
| 1:62598774:CA:C | frameshift | c.575delA | p.Gln192fs | Yes | Yes | No | No |
| 1:62598787:T:C | missense (5/5) | c.587T > C | p.Ile196Thr | Yes | No | No | No |
| 1:62598798:GA:G | frameshift | c.602delA | p.Asn201fs | Yes | Yes | No | No |
| 1:62598799:A:G | missense (5/5) | c.599A > G | p.Glu200Gly | Yes | No | No | No |
| 1:62598807:G:A | splice donor | c.606 + 1G > A | | Yes | Yes | No | No |
| 1:62598807:G:C | splice donor | c.606 + 1G > C | | Yes | Yes | No | No |
| 1:62601080:A:G | spliceacceptor | c.607 − 2A > G | | Yes | Yes | No | No |
| 1:62601115:AT:A | frameshift | c.643delT | p.Ser215fs | Yes | Yes | No | No |
| 1:62601197:G:A | splice donor | c.721 + 1G > A | | Yes | Yes | No | No |
| 1:62601783:T:G | missense (5/5) | c.736T > G | p.Cys246Gly | Yes | No | No | No |
| 1:62601796:A:G | missense (5/5) | c.749A > G | p.Tyr250Cys | Yes | No | No | No |
| 1:62601804:G:T | missense (5/5) | c.757G > T | p.Gly253Cys | Yes | No | No | No |
| 1:62601855:T:C | missense (5/5) | c.808T > C | p.Phe270Leu | Yes | No | No | No |
| 1:62601861:G:T | missense (5/5) | c.814G > T | p.Val272Phe | Yes | No | No | No |
| 1:62601868:G:A | missense (5/5) | c.821G > A | p.Cys274Tyr | Yes | No | No | No |
| 1:62601868:G:T | missense (5/5) | c.821G > T | p.Cys274Phe | Yes | No | No | No |
| 1:62601870:GATGTT:G | frameshift | c.826_830delGTTAT | p.Val276fs | Yes | Yes | No | No |
| 1:62601880:C:G | stop gained | c.833C > G | p.Ser278* | Yes | Yes | No | No |
| 1:62601880:C:A | stop gained | c.833C > A | p.Ser278* | Yes | Yes | No | No |
| 1:62602284:G:C | splice acceptor | c.836 − 1G > C | | Yes | Yes | No | No |
| 1:62602297:C:A | missense (5/5) | c.848C > A | p.Thr283Lys | Yes | No | No | No |
| 1:62602300:T:C | missense (5/5) | c.851T > C | p.Leu284Ser | Yes | No | No | No |
| 1:62602306:A:G | missense (5/5) | c.857A > G | p.Gln286Arg | Yes | No | No | No |
| 1:62602311:C:T | stop gained | c.862C > T | p.Arg288* | Yes | Yes | No | No |
| 1:62602316:AGATG:A | frameshift | c.871_874delGGAT | p.Gly291fs | Yes | Yes | No | No |
| 1:62602317:G:C | missense (5/5) | c.868G > C | p.Asp290His | Yes | No | No | No |
| 1:62602323:T:C | missense (5/5) | c.874T > C | p.Ser292Pro | Yes | No | No | No |
| 1:62602331:C:A | missense (5/5) | c.882C > A | p.Asn294Lys | Yes | No | No | No |
| 1:62602332:T:G | missense (5/5) | c.883T > G | p.Phe295Val | Yes | No | No | No |
| 1:62602337:T:A | missense (5/5) | c.888T > A | p.Asn296Lys | Yes | No | No | No |
| 1:62602338:GA:G | frameshift | c.892delA | p.Thr298fs | Yes | Yes | No | No |
| 1:62602342:C:T | missense (5/5) | c.893C > T | p.Thr298Met | Yes | No | No | No |
| 1:62602345:G:A | stop gained | c.896G > A | p.Trp299* | Yes | Yes | No | No |
| 1:62602346:G:A | stop gained | c.897G > A | p.Trp299* | Yes | Yes | No | No |
| 1:62602355:C:A | stop gained | c.906C > A | p.Tyr302* | Yes | Yes | No | No |
| 1:62602373:GC:G | frameshift | c.925delC | p.Asp310fs | Yes | Yes | No | No |
| 1:62602381:G:GT | splice donor | c.931 + 1_931 + 2insT | | Yes | Yes | No | No |
| 1:62602381:G:A | splice donor | c.931 + 1G > A | | Yes | Yes | No | No |
| 1:62603986:C:CT | frameshift | c.950dupT | p.Glu318fs | Yes | Yes | No | No |
| 1:62603993:A:G | missense (5/5) | c.956A > G | p.Lys319Arg | Yes | No | No | No |
| 1:62603996:T:C | missense (5/5) | c.959T > C | p.Ile320Thr | Yes | No | No | No |
| 1:62604005:T:C | missense (5/5) | c.968T > C | p.Ile323Thr | Yes | No | No | No |
| 1:62604017:CTAAT:C | frameshift | c.983_986delATTA | p.Asn328fs | Yes | Yes | No | No |
| 1:62604024:T:A | stop gained | c.987T > A | p.Tyr329* | Yes | Yes | No | No |
| 1:62604031:C:T | stop gained | c.994C > T | p.Arg332* | Yes | Yes | No | No |
| 1:62604035:T:G | missense (5/5) | c.998T > G | p.Ile333Ser | Yes | No | No | No |
| 1:62604035:T:A | missense (5/5) | c.998T > A | p.Ile333Asn | Yes | No | No | No |
| 1:62604037:G:T | stop gained | c.1000G > T | p.Glu334* | Yes | Yes | No | No |
| 1:62604044:A:G | missense (5/5) | c.1007A > G | p.Glu336Gly | Yes | No | No | No |
| 1:62604049:T:C | missense (5/5) | c.1012T > C | p.Trp338Arg | Yes | No | No | No |
| 1:62604051:G:C | missense (5/5) | c.1014G > C | p.Trp338Cys | Yes | No | No | No |
| 1:62604083:T:G | missense (5/5) | c.1046T > G | p.Phe349Cys | Yes | No | No | No |
| 1:62604109:T:C | missense (5/5) | c.1072T > C | p.Tyr358His | Yes | No | No | No |
| 1:62604160:GA:G | frameshift | c.1127delA | p.Asn376fs | Yes | Yes | No | No |
| 1:62604170:AT:A | frameshift | c.1136delT | p.Leu379fs | Yes | Yes | No | No |
| 1:62604184:A:T | missense (5/5) | c.1147A > T | p.Thr383Ser | Yes | No | No | No |
| 1:62604185:C:T | missense (5/5) | c.1148C > T | p.Thr383Ile | Yes | No | No | No |
| 1:62604187:TG:T | frameshift | c.1153delG | p.Asp385fs | Yes | Yes | No | No |
| 1:62604190:G:C | missense (5/5) | c.1153G > C | p.Asp385His | Yes | No | No | No |
| 1:62604190:G:T | missense (5/5) | c.1153G > T | p.Asp385Tyr | Yes | No | No | No |
| 1:62604218:G:C | missense (5/5) | c.1181G > C | p.Cys394Ser | Yes | No | No | No |
| 1:62604224:A:AGG | frameshift | c.1189_1190dupGG | p.Tyr398fs | Yes | Yes | No | No |
| 1:62604233:C:A | stop gained | c.1196C > A | p.Ser399* | Yes | Yes | No | No |
| 1:62604235:G:A | missense (5/5) | c.1198G > A | p.Gly400Arg | Yes | No | No | No |
| 1:62604236:G:T | splice donor | c.1198 + 1G > T | | Yes | Yes | No | No |
| 1:62604639:G:A | stop gained | c.1205G > A | p.Trp402* | Yes | Yes | No | No |
| 1:62604640:G:GT | frameshift | c.1207dupT | p.Trp403fs | Yes | Yes | No | No |
| 1:62604645:G:A | stop gained | c.1211G > A | p.Trp404* | Yes | Yes | No | No |
| 1:62604656:T:C | missense (5/5) | c.1222T > C | p.Cys408Arg | Yes | No | No | No |
| 1:62604669:AC:A | frameshift | c.1237delC | p.Leu413fs | Yes | Yes | No | No |
| 1:62604684:A:G | missense (5/5) | c.1250A > G | p.Tyr417Cys | Yes | No | No | No |
| 1:62604701:A:C | missense (5/5) | c.1267A > C | p.Lys423Gln | Yes | No | No | No |
| 1:62604762:C:CT | frameshift | c.1329dupT | p.Ile444fs | Yes | Yes | No | No |
| 1:62604780:T:G | missense (5/5) | c.1346T > G | p.Met449Arg | Yes | No | No | No |
| 1:62604803:G:T | stop gained | c.1369G > T | p.Glu457* | Yes | Yes | No | No |
| 1:62604806:AG:A | frameshift | c.1373delG | p.Ser458fs | Yes | Yes | No | No |
| 1:62604809:T:TGAATCTG | frameshift | c.1375_1376insGAATCTG | p.Phe459fs | Yes | Yes | No | No |
| 16:31483138:T:C | start lost | c.2T > C | p.Met1? | No | No | Yes | Yes |
| 16:31483139:G:C | start lost | c.3G > C | p.Met1? | No | No | Yes | Yes |
| 16:31483166:A:AC | frameshift | c.32dupC | p.Glu12fs | No | No | Yes | Yes |
| 16:31483174:TG:T | frameshift | c.43delG | p.Ala15fs | No | No | Yes | Yes |
| 16:31483195:T:C | missense (5/5) | c.59T > C | p.Ile20Thr | No | No | Yes | No |
| 16:31483235:C:A | missense (5/5) | c.99C > A | p.Phe33Leu | No | No | Yes | No |
| 16:31483252:T:C | missense (5/5) | c.116T > C | p.Val39Ala | No | No | Yes | No |
| 16:31483260:T:G | missense (5/5) | c.124T > G | p.Trp42Gly | No | No | Yes | No |
| 16:31483262:G:A | stop gained | c.126G > A | p.Trp42* | No | No | Yes | Yes |
| 16:31483263:G:A | splice donor | c.126 + 1G > A | | No | No | Yes | Yes |
| 16:31484674:C:T | missense (5/5) | c.128C > T | p.Ser43Phe | No | No | Yes | No |
| 16:31484682:A:G | missense (5/5) | c.136A > G | p.Arg46Gly | No | No | Yes | No |
| 16:31484697:A:C | missense (5/5) | c.151A > C | p.Thr51Pro | No | No | Yes | No |
| 16:31484706:G:C | missense (5/5) | c.160G > C | p.Gly54Arg | No | No | Yes | No |
| 16:31484706:G:A | missense (5/5) | c.160G > A | p.Gly54Ser | No | No | Yes | No |
| 16:31484707:G:A | missense (5/5) | c.161G > A | p.Gly54Asp | No | No | Yes | No |
| 16:31484710:A:C | missense (5/5) | c.164A > C | p.Tyr55Ser | No | No | Yes | No |
| 16:31484714:C:G | missense (5/5) | c.168C > G | p.Phe56Leu | No | No | Yes | No |
| 16:31484716:T:C | missense (5/5) | c.170T > C | p.Leu57Pro | No | No | Yes | No |
| 16:31484719:C:A | missense (5/5) | c.173C > A | p.Ala58Glu | No | No | Yes | No |
| 16:31484724:C:T | missense (5/5) | c.178C > T | p.Arg60Cys | No | No | Yes | No |
| 16:31484725:G:C | missense (5/5) | c.179G > C | p.Arg60Pro | No | No | Yes | No |
| 16:31484725:G:A | missense (5/5) | c.179G > A | p.Arg60His | No | No | Yes | No |
| 16:31484731:TG:T | frameshift | c.187delG | p.Val63fs | No | No | Yes | Yes |
| 16:31484732:G:T | missense (5/5) | c.186G > T | p.Met62Ile | No | No | Yes | No |
| 16:31484738:G:T | missense (5/5) | c.192G > T | p.Trp64Cys | No | No | Yes | No |
| 16:31484740:G:A | stop gained | c.194G > A | p.Trp65* | No | No | Yes | Yes |
| 16:31484743:C:A | missense (5/5) | c.197C > A | p.Pro66Gln | No | No | Yes | No |
| 16:31484817:A:G | spliceacceptor | c.199 − 2A > G | | No | No | Yes | Yes |
| 16:31484817:AG:A | frameshift | c.199delG | p.Val67fs | No | No | Yes | Yes |
| 16:31484826:CCT:C | frameshift | c.213_214delCT | p.Phe72fs | No | No | Yes | Yes |
| 16:31484828:T:A | missense (5/5) | c.208T > A | p.Ser70Thr | No | No | Yes | No |
| 16:31484831:C:G | missense (5/5) | c.211C > G | p.Leu71Val | No | No | Yes | No |
| 16:31484836:C:CAG | frameshift | c.216_217insAG | p.Ala73fs | No | No | Yes | Yes |
| 16:31484837:G:A | missense (5/5) | c.217G > A | p.Ala73Thr | No | No | Yes | No |
| 16:31484849:G:A | missense (5/5) | c.229G > A | p.Gly77Ser | No | No | Yes | No |
| 16:31484849:G:T | missense (5/5) | c.229G > T | p.Gly77Cys | No | No | Yes | No |
| 16:31484849:G:C | missense (5/5) | c.229G > C | p.Gly77Arg | No | No | Yes | No |
| 16:31484864:G:T | missense (5/5) | c.244G > T | p.Val82Leu | No | No | Yes | No |
| 16:31484864:G:A | missense (5/5) | c.244G > A | p.Val82Met | No | No | Yes | No |
| 16:31484865:T:G | missense (5/5) | c.245T > G | p.Val82Gly | No | No | Yes | No |
| 16:31484868:G:A | missense (5/5) | c.248G > A | p.Gly83Asp | No | No | Yes | No |
| 16:31484870:C:G | missense (5/5) | c.250C > G | p.Leu84Val | No | No | Yes | No |
| 16:31484877:G:A | missense (5/5) | c.257G > A | p.Gly86Glu | No | No | Yes | No |
| 16:31484885:G:A | missense (5/5) | c.265G > A | p.Ala89Thr | No | No | Yes | No |
| 16:31484888:G:A | missense (5/5) | c.268G > A | p.Ala90Thr | No | No | Yes | No |
| 16:31484914:C:CTT | frameshift | c.294_295insTT | p.Glu99fs | No | No | Yes | Yes |
| 16:31484915:G:A | missense (5/5) | c.295G > A | p.Glu99Lys | No | No | Yes | No |
| 16:31484919:G:A | stop gained | c.299G > A | p.Trp100* | No | No | Yes | Yes |
| 16:31485730:C:T | missense (5/5) | c.305C > T | p.Ala102Val | No | No | Yes | No |
| 16:31485745:T:G | missense (5/5) | c.320T > G | p.Leu107Arg | No | No | Yes | No |
| 16:31485745:T:C | missense (5/5) | c.320T > C | p.Leu107Pro | No | No | Yes | No |
| 16:31485748:T:C | missense (5/5) | c.323T > C | p.Leu108Pro | No | No | Yes | No |
| 16:31485751:T:G | missense (5/5) | c.326T > G | p.Leu109Arg | No | No | Yes | No |
| 16:31485753:G:A | missense (5/5) | c.328G > A | p.Gly110Ser | No | No | Yes | No |
| 16:31485768:C:T | missense (5/5) | c.343C > T | p.Pro115Ser | No | No | Yes | No |
| 16:31485771:G:A | missense (5/5) | c.346G > A | p.Val116Met | No | No | Yes | No |
| 16:31485771:G:T | missense (5/5) | c.346G > T | p.Val116Leu | No | No | Yes | No |
| 16:31485774:T:C | missense (5/5) | c.349T > C | p.Tyr117His | No | No | Yes | No |
| 16:31485775:A:G | missense (5/5) | c.350A > G | p.Tyr117Cys | No | No | Yes | No |
| 16:31485784:C:T | missense (5/5) | c.359C > T | p.Ala120Val | No | No | Yes | No |
| 16:31485786:G:T | missense (5/5) | c.361G > T | p.Gly121Trp | No | No | Yes | No |
| 16:31485793:T:A | missense (5/5) | c.368T > A | p.Ile123Asn | No | No | Yes | No |
| 16:31485796:C:T | missense (5/5) | c.371C > T | p.Thr124Met | No | No | Yes | No |
| 16:31485799:T:C | missense (5/5) | c.374T > C | p.Met125Thr | No | No | Yes | No |
| 16:31485806:G:T | missense (5/5) | c.381G > T | p.Gln127His | No | No | Yes | No |
| 16:31485809:C:G | stop gained | c.384C > G | p.Tyr128* | No | No | Yes | Yes |
| 16:31485811:T:C | missense (5/5) | c.386T > C | p.Leu129Pro | No | No | Yes | No |
| 16:31485813:C:T | missense (5/5) | c.388C > T | p.Arg130Cys | No | No | Yes | No |
| 16:31485819:C:T | missense (5/5) | c.394C > T | p.Arg132Cys | No | No | Yes | No |
| 16:31485819:C:A | missense (5/5) | c.394C > A | p.Arg132Ser | No | No | Yes | No |
| 16:31485820:G:A | missense (5/5) | c.395G > A | p.Arg132His | No | No | Yes | No |
| 16:31485824:C:A | missense (5/5) | c.399C > A | p.Phe133Leu | No | No | Yes | No |
| 16:31485825:G:C | missense (5/5) | c.400G > C | p.Gly134Arg | No | No | Yes | No |
| 16:31485825:G:A | missense (5/5) | c.400G > A | p.Gly134Ser | No | No | Yes | No |
| 16:31485828:G:A | missense (5/5) | c.403G > A | p.Gly135Ser | No | No | Yes | No |
| 16:31485834:C:G | missense (5/5) | c.409C > G | p.Arg137Gly | No | No | Yes | No |
| 16:31485834:C:T | missense (5/5) | c.409C > T | p.Arg137Cys | No | No | Yes | No |
| 16:31485835:G:A | missense (5/5) | c.410G > A | p.Arg137His | No | No | Yes | No |
| 16:31485837:A:T | missense (5/5) | c.412A > T | p.Ile138Phe | No | No | Yes | No |
| 16:31485840:C:T | missense (5/5) | c.415C > T | p.Arg139Cys | No | No | Yes | No |
| 16:31485841:G:T | missense (5/5) | c.416G > T | p.Arg139Leu | No | No | Yes | No |
| 16:31485841:G:A | missense (5/5) | c.416G > A | p.Arg139His | No | No | Yes | No |
| 16:31485848:C:A | stop gained | c.423C > A | p.Tyr141* | No | No | Yes | Yes |
| 16:31485850:T:G | missense (5/5) | c.425T > G | p.Leu142Arg | No | No | Yes | No |
| 16:31485852:T:C | missense (5/5) | c.427T > C | p.Ser143Pro | No | No | Yes | No |
| 16:31485865:T:A | missense (5/5) | c.440T > A | p.Leu147His | No | No | Yes | No |
| 16:31485873:T:A | missense (5/5) | c.448T > A | p.Tyr150Asn | No | No | Yes | No |
| 16:31485873:T:C | missense (5/5) | c.448T > C | p.Tyr150His | No | No | Yes | No |
| 16:31485876:A:C | missense (5/5) | c.451A > C | p.Ile151LeU | No | No | Yes | No |
| 16:31485885:A:G | missense (5/5) | c.460A > G | p.Lys154Glu | No | No | Yes | No |
| 16:31485894:G:C | splice donor | c.468 + 1G > C | | No | No | Yes | Yes |
| 16:31486168:A:T | splice acceptor | c.469 − 2A > T | | No | No | Yes | Yes |
| 16:31486177:T:G | missense (5/5) | c.476T > G | p.Met159Arg | No | No | Yes | No |
| 16:31486182:T:C | missense (5/5) | c.481T > C | p.Ser161Pro | No | No | Yes | No |
| 16:31486183:C:T | missense (5/5) | c.482C > T | p.Ser161Phe | No | No | Yes | No |
| 16:31486185:G:A | missense (5/5) | c.484G > A | p.Gly162Arg | No | No | Yes | No |
| 16:31486200:CA:C | frameshift | c.500delA | p.Gln167fs | No | No | Yes | Yes |
| 16:31486204:A:G | missense (5/5) | c.503A > G | p.Gln168Arg | No | No | Yes | No |
| 16:31486205:G:T | missense (5/5) | c.504G > T | p.Gln168His | No | No | Yes | No |
| 16:31486206:GC:G | frameshift | c.506delC | p.Ala169fs | No | No | Yes | Yes |
| 16:31486207:C:A | missense (5/5) | c.506C > A | p.Ala169Asp | No | No | Yes | No |
| 16:31486210:T:C | missense (5/5) | c.509T > C | p.Leu170Pro | No | No | Yes | No |
| 16:31486212:G:A | missense (5/5) | c.511G > A | p.Gly171Ser | No | No | Yes | No |
| 16:31486218:A:C | missense (5/5) | c.517A > C | p.Asn173His | No | No | Yes | No |
| 16:31486222:T:C | missense (5/5) | c.521T > C | p.Ile174Thr | No | No | Yes | No |
| 16:31486223:C:G | missense (5/5) | c.522C > G | p.Ile174Met | No | No | Yes | No |
| 16:31486225:A:C | missense (5/5) | c.524A > C | p.Tyr175Ser | No | No | Yes | No |
| 16:31486231:C:T | missense (5/5) | c.530C > T | p.Ser177Phe | No | No | Yes | No |
| 16:31486240:C:A | missense (5/5) | c.539C > A | p.Ala180Glu | No | No | Yes | No |
| 16:31486242:C:T | missense (5/5) | c.541C > T | p.Leu181Phe | No | No | Yes | No |
| 16:31486252:T:A | missense (5/5) | c.551T > A | p.Ile184Asn | No | No | Yes | No |
| 16:31486267:C:T | missense (5/5) | c.566C > T | p.Thr189Met | No | No | Yes | No |
| 16:31486269:G:A | missense (5/5) | c.568G > A | p.Val190Met | No | No | Yes | No |
| 16:31486272:A:C | missense (5/5) | c.571A > C | p.Thr191Pro | No | No | Yes | No |
| 16:31487318:A:G | splice acceptor | c.575 − 2A > G | | No | No | Yes | Yes |
| 16:31487322:G:A | missense (5/5) | c.577G > A | p.Gly193Arg | No | No | Yes | No |
| 16:31487323:G:A | missense (5/5) | c.578G > A | p.Gly193GlU | No | No | Yes | No |
| 16:31487328:G:C | missense (5/5) | c.583G > C | p.Ala195Pro | No | No | Yes | No |
| 16:31487341:A:G | missense (5/5) | c.596A > G | p.Tyr199Cys | No | No | Yes | No |
| 16:31487343:A:G | missense (5/5) | c.598A > G | p.Thr200Ala | No | No | Yes | No |
| 16:31487355:C:T | stop gained | c.610C > T | p.Gln204* | No | No | Yes | Yes |
| 16:31487532:T:G | missense (5/5) | c.658T > G | p.Phe220Val | No | No | Yes | No |
| 16:31487544:G:A | missense (5/5) | c.670G > A | p.Gly224Ser | No | No | Yes | No |
| 16:31487547:G:C | missense (5/5) | c.673G > C | p.Gly225Arg | No | No | Yes | No |
| 16:31487547:G:A | missense (5/5) | c.673G > A | p.Gly225Arg | No | No | Yes | No |
| 16:31487557:G:T | missense (5/5) | c.683G > T | p.Gly228Val | No | No | Yes | No |
| 16:31487571:T:C | missense (5/5) | c.697T > C | p.Tyr233His | No | No | Yes | No |
| 16:31487571:T:TA | frameshift | c.698dupA | p.Tyr233fs | No | No | Yes | Yes |
| 16:31487596:C:CG | frameshift | c.724dupG | p.Val242fs | No | No | Yes | Yes |
| 16:31487607:G:C | missense (5/5) | c.733G > C | p.Asp245His | No | No | Yes | No |
| 16:31487635:TC:T | frameshift | c.762delC | p.Cys255fs | No | No | Yes | Yes |
| 16:31487642:T:A | stop gained | c.768T > A | p.Tyr256* | No | No | Yes | Yes |
| 16:31487642:T:G | stop gained | c.768T > G | p.Tyr256* | No | No | Yes | Yes |
| 16:31487655:G:T | missense (5/5) | c.781G > T | p.Asp261Tyr | No | No | Yes | No |
| 16:31487659:C:G | missense (5/5) | c.785C > G | p.Ser262Cys | No | No | Yes | No |
| 16:31487673:C:T | missense (5/5) | c.799C > T | p.Arg267Trp | No | No | Yes | No |
| 16:31487674:G:A | missense (5/5) | c.800G > A | p.Arg267Gln | No | No | Yes | No |
| 16:31487688:G:A | missense (5/5) | c.814G > A | p.Gly272Arg | No | No | Yes | No |
| 16:31487698:C:T | missense (5/5) | c.824C > T | p.Pro275Leu | No | No | Yes | No |
| 16:31487698:C:G | missense (5/5) | c.824C > G | p.Pro275Arg | No | No | Yes | No |
| 16:31487718:G:A | missense (5/5) | c.844G > A | p.Gly282Arg | No | No | Yes | No |
| 16:31487718:G:C | missense (5/5) | c.844G > C | p.Gly282Arg | No | No | Yes | No |
| 16:31487722:TCA:T | frameshift | c.851_852delCA | p.Thr284fs | No | No | Yes | Yes |
| 16:31487723:C:CA | frameshift | c.850dupA | p.Thr284fs | No | No | Yes | Yes |
| 16:31487725:C:T | missense (5/5) | c.851C > T | p.Thr284Ile | No | No | Yes | No |
| 16:31487754:G:T | missense (5/5) | c.880G > T | p.Asp294Tyr | No | No | Yes | No |
| 16:31488032:C:CCGTA | splice acceptor | c.886 − 5_886 − 2dupCGTA | | No | No | Yes | Yes |
| 16:31488038:G:C | missense (5/5) | c.886G > C | p.Val296Leu | No | No | Yes | No |
| 16:31488041:A:G | missense (5/5) | c.889A > G | p.Ile297Val | No | No | Yes | No |
| 16:31488042:T:C | missense (5/5) | c.890T > C | p.Ile297Thr | No | No | Yes | No |
| 16:31488044:G:A | missense (5/5) | c.892G > A | p.Val298Met | No | No | Yes | No |
| 16:31488045:T:C | missense (5/5) | c.893T > C | p.Val298Ala | No | No | Yes | No |
| 16:31488050:C:G | missense (5/5) | c.898C > G | p.Arg300Gly | No | No | Yes | No |
| 16:31488050:C:T | missense (5/5) | c.898C > T | p.Arg300Cys | No | No | Yes | No |
| 16:31488051:G:C | missense (5/5) | c.899G > C | p.Arg300Pro | No | No | Yes | No |
| 16:31488053:T:C | missense (5/5) | c.901T > C | p.Cys301Arg | No | No | Yes | No |
| 16:31488057:T:C | missense (5/5) | c.905T > C | p.Leu302Pro | No | No | Yes | No |
| 16:31488062:G:A | missense (5/5) | c.910G > A | p.Gly304Arg | No | No | Yes | No |
| 16:31488062:G:C | missense (5/5) | c.910G > C | p.Gly304Arg | No | No | Yes | No |
| 16:31488077:C:A | missense (5/5) | c.925C > A | p.His309Asn | No | No | Yes | No |
| 16:31488083:A:G | missense (5/5) | c.931A > G | p.Lys311Glu | No | No | Yes | No |
| 16:31488086:G:A | missense (5/5) | c.934G > A | p.Ala312Thr | No | No | Yes | No |
| 16:31488087:C:A | missense (5/5) | c.935C > A | p.Ala312Glu | No | No | Yes | No |
| 16:31488087:C:T | missense (5/5) | c.935C > T | p.Ala312Val | No | No | Yes | No |
| 16:31488092:T:C | missense (5/5) | c.940T > C | p.Cys314Arg | No | No | Yes | No |
| 16:31488102:G:A | missense (5/5) | c.950G > A | p.Cys317Tyr | No | No | Yes | No |
| 16:31488107:T:TA | frameshift | c.956dupA | p.Tyr319fs | No | No | Yes | Yes |
| 16:31488109:C:A | stop gained | c.957C > A | p.Tyr319* | No | No | Yes | Yes |
| 16:31488109:C:G | stop gained | c.957C > G | p.Tyr319* | No | No | Yes | Yes |
| 16:31488114:A:G | missense (5/5) | c.962A > G | p.Lys321Arg | No | No | Yes | No |
| 16:31488122:C:A | missense (5/5) | c.970C > A | p.Pro324Thr | No | No | Yes | No |
| 16:31488126:T:C | missense (5/5) | c.974T > C | p.Met325Thr | No | No | Yes | No |
| 16:31488134:A:G | missense (5/5) | c.982A > G | p.Met328Val | No | No | Yes | No |
| 16:31488135:T:G | missense (5/5) | c.983T > G | p.Met328Arg | No | No | Yes | No |
| 16:31488135:T:C | missense (5/5) | c.983T > C | p.Met328Thr | No | No | Yes | No |
| 16:31488138:T:C | missense (5/5) | c.986T > C | p.Val329Ala | No | No | Yes | No |
| 16:31488146:G:C | missense (5/5) | c.994G > C | p.Gly332Arg | No | No | Yes | No |
| 16:31488146:G:A | missense (5/5) | c.994G > A | p.Gly332Ser | No | No | Yes | No |
| 16:31488147:G:C | missense (5/5) | c.995G > C | p.Gly332Ala | No | No | Yes | No |
| 16:31488151:G:T | missense (5/5) | c.999G > T | p.Met333Ile | No | No | Yes | No |
| 16:31488151:G:A | missense (5/5) | c.999G > A | p.Met333Ile | No | No | Yes | No |
| 16:31488157:C:A | missense (5/5) | c.1005C > A | p.Ser335Arg | No | No | Yes | No |
| 16:31488158:C:T | missense (5/5) | c.1006C > T | p.Arg336Cys | No | No | Yes | No |
| 16:31488159:G:T | missense (5/5) | c.1007G > T | p.Arg336Leu | No | No | Yes | No |
| 16:31488159:G:A | missense (5/5) | c.1007G > A | p.Arg336His | No | No | Yes | No |
| 16:31488169:C:G | stop gained | c.1017C > G | p.Tyr339* | No | No | Yes | Yes |
| 16:31488170:C:T | missense (5/5) | c.1018C > T | p.Pro340Ser | No | No | Yes | No |
| 16:31488388:G:GTGGC | frameshift | c.1028_1031dupTGGC | p.Cys345fs | No | No | Yes | Yes |
| 16:31488392:C:T | missense (5/5) | c.1031C > T | p.Ala344Val | No | No | Yes | No |
| 16:31488397:G:A | missense (5/5) | c.1036G > A | p.Val346Met | No | No | Yes | No |
| 16:31488403:C:A | missense (5/5) | c.1042C > A | p.Pro348Thr | No | No | Yes | No |
| 16:31488412:T:C | missense (5/5) | c.1051T > C | p.Cys351Arg | No | No | Yes | No |
| 16:31488419:GC:G | frameshift | c.1059delC | p.Val354fs | No | No | Yes | Yes |
| 16:31488424:T:C | missense (5/5) | c.1063T > C | p.Cys355Arg | No | No | Yes | No |
| 16:31488426:C:A | stop gained | c.1065C > A | p.Cys355* | No | No | Yes | Yes |
| 16:31488446:C:T | missense (5/5) | c.1085C > T | p.Ser362Phe | No | No | Yes | No |
| 16:31488449:A:T | missense (5/5) | c.1088A > T | p.Asn363Ile | No | No | Yes | No |
| 16:31488454:G:A | missense (5/5) | c.1093G > A | p.Ala365Thr | No | No | Yes | No |
| 16:31488459:C:G | stop gained | c.1098C > G | p.Tyr366* | No | No | Yes | Yes |
| 16:31488463:C:T | missense (5/5) | c.1102C > T | p.Arg368Trp | No | No | Yes | No |
| 16:31488469:G:A | missense (5/5) | c.1108G > A | p.Val370Ile | No | No | Yes | No |
| 16:31488470:T:C | missense (5/5) | c.1109T > C | p.Val370Ala | No | No | Yes | No |
| 16:31488473:T:A | missense (5/5) | c.1112T > A | p.Val371Glu | No | No | Yes | No |
| 16:31488473:T:G | missense (5/5) | c.1112T > G | p.Val371Gly | No | No | Yes | No |
| 16:31488478:C:T | missense (5/5) | c.1117C > T | p.Leu373Phe | No | No | Yes | No |
| 16:31488483:G:A | missense (5/5) | c.1122G > A | p.Met374Ile | No | No | Yes | No |
| 16:31488484:C:G | missense (5/5) | c.1123C > G | p.Pro375Ala | No | No | Yes | No |
| 16:31488485:C:T | missense (5/5) | c.1124C > T | p.Pro375Leu | No | No | Yes | No |
| 16:31488491:G:T | splice donor | c.1129 + 1G > T | | No | No | Yes | Yes |
| 16:31488620:A:C | splice acceptor | c.1130 − 2A > C | | No | No | Yes | Yes |
| 16:31488621:G:A | splice acceptor | c.1130 − 1G > A | | No | No | Yes | Yes |
| 16:31488622:G:A | missense (5/5) | c.1130G > A | p.Gly377Asp | No | No | Yes | No |
| 16:31488622:G:C | missense (5/5) | c.1130G > C | p.Gly377Ala | No | No | Yes | No |
| 16:31488625:T:C | missense (5/5) | c.1133T > C | p.Leu378Pro | No | No | Yes | No |
| 16:31488629:C:CG | frameshift | c.1139dupG | p.Leu381fs | No | No | Yes | Yes |
| 16:31488631:G:A | missense (5/5) | c.1139G > A | p.Gly380Glu | No | No | Yes | No |
| 16:31488636:A:C | missense (5/5) | c.1144A > C | p.Met382Leu | No | No | Yes | No |
| 16:31488637:T:A | missense (5/5) | c.1145T > A | p.Met382Lys | No | No | Yes | No |
| 16:31488637:T:C | missense (5/5) | c.1145T > C | p.Met382Thr | No | No | Yes | No |
| 16:31488638:G:A | missense (5/5) | c.1146G > A | p.Met382Ile | No | No | Yes | No |
| 16:31488640:T:C | missense (5/5) | c.1148T > C | p.Leu383Pro | No | No | Yes | No |
| 16:31488643:C:T | missense (5/5) | c.1151C > T | p.Ala384Val | No | No | Yes | No |
| 16:31488645:G:A | missense (5/5) | c.1153G > A | p.Val385Ile | No | No | Yes | No |
| 16:31488645:G:T | missense (5/5) | c.1153G > T | p.Val385Phe | No | No | Yes | No |
| 16:31488648:A:C | missense (5/5) | c.1156A > C | p.Met386Leu | No | No | Yes | No |
| 16:31488649:T:C | missense (5/5) | c.1157T > C | p.Met386Thr | No | No | Yes | No |
| 16:31488657:G:A | missense (5/5) | c.1165G > A | p.Ala389Thr | No | No | Yes | No |
| 16:31488663:A:G | missense (5/5) | c.1171A > G | p.Met391Val | No | No | Yes | No |
| 16:31488664:T:A | missense (5/5) | c.1172T > A | p.Met391Lys | No | No | Yes | No |
| 16:31488664:T:C | missense (5/5) | c.1172T > C | p.Met391Thr | No | No | Yes | No |
| 16:31488666:T:TCC | frameshift | c.1175_1176dupCC | p.Ser393fs | No | No | Yes | Yes |
| 16:31488676:C:T | missense (5/5) | c.1184C > T | p.Ala395Val | No | No | Yes | No |
| 16:31488677:C:CTCCA | frameshift | c.1188_1191dupCATC | p.Phe398fs | No | No | Yes | Yes |
| 16:31488679:C:T | missense (5/5) | c.1187C > T | p.Ser396Phe | No | No | Yes | No |
| 16:31488700:C:G | missense (5/5) | c.1208C > G | p.Thr403Arg | No | No | Yes | No |
| 16:31488712:T:C | missense (5/5) | c.1220T > C | p.Met407Thr | No | No | Yes | No |
| 16:31488714:G:T | missense (5/5) | c.1222G > T | p.Asp408Tyr | No | No | Yes | No |
| 16:31488717:A:T | missense (5/5) | c.1225A > T | p.Ile409Phe | No | No | Yes | No |
| 16:31488719:C:G | missense (5/5) | c.1227C > G | p.Ile409Met | No | No | Yes | No |
| 16:31488721:A:ACACGCGCCTGCGGC | frameshift | c.1238_1251dupTGCGGCCACGCGCC | p.Gly418fs | No | No | Yes | Yes |
| 16:31488722:C:G | stop gained | c.1230C > G | p.Tyr410* | No | No | Yes | Yes |
| 16:31488732:C:T | missense (5/5) | c.1240C > T | p.Arg414Trp | No | No | Yes | No |
| 16:31488733:G:A | missense (5/5) | c.1241G > A | p.Arg414Gln | No | No | Yes | No |
| 16:31488753:G:A | missense (5/5) | c.1261G > A | p.Glu421Lys | No | No | Yes | No |
| 16:31488755:G:C | missense (5/5) | c.1263G > C | p.Glu421Asp | No | No | Yes | No |
| 16:31488765:G:A | missense (5/5) | c.1273G > A | p.Val425Met | No | No | Yes | No |
| 16:31488773:G:A | splice donor | c.1280 + 1G > A | | No | No | Yes | Yes |
| 16:31488878:A:C | splice acceptor | c.1281 − 2A > C | | No | No | Yes | Yes |
| 16:31488882:T:G | missense (5/5) | c.1283T > G | p.Leu428Arg | No | No | Yes | No |
| 16:31488884:T:C | missense (5/5) | c.1285T > C | p.Trp429Arg | No | No | Yes | No |
| 16:31488897:T:A | missense (5/5) | c.1298T > A | p.Ile433Asn | No | No | Yes | No |
| 16:31488897:T:C | missense (5/5) | c.1298T > C | p.Ile433Thr | No | No | Yes | No |
| 16:31488899:G:A | missense (5/5) | c.1300G > A | p.Val434Met | No | No | Yes | No |
| 16:31488900:T:A | missense (5/5) | c.1301T > A | p.Val434Glu | No | No | Yes | No |
| 16:31488900:T:C | missense (5/5) | c.1301T > C | p.Val434Ala | No | No | Yes | No |
| 16:31488911:GTGGCC:G | frameshift | c.1317_1321delCTGGC | p.Trp440fs | No | No | Yes | Yes |
| 16:31488917:T:C | missense (5/5) | c.1318T > C | p.Trp440Arg | No | No | Yes | No |
| 16:31488918:G:A | stop gained | c.1319G > A | p.Trp440* | No | No | Yes | Yes |
| 16:31488923:C:T | missense (5/5) | c.1324C > T | p.Pro442Ser | No | No | Yes | No |
| 16:31488934:G:C | missense (5/5) | c.1335G > C | p.Gln445His | No | No | Yes | No |
| 16:31488942:A:G | missense (5/5) | c.1343A > G | p.Gln448Arg | No | No | Yes | No |
| 16:31488942:A:T | missense (5/5) | c.1343A > T | p.Gln448Leu | No | No | Yes | No |
| 16:31488942:A:AG | frameshift | c.1346dupG | p.Gly450fs | No | No | Yes | Yes |
| 16:31488945:G:A | missense (5/5) | c.1346G > A | p.Gly449Asp | No | No | Yes | No |
| 16:31488947:G:A | missense (5/5) | c.1348G > A | p.Gly450Arg | No | No | Yes | No |
| 16:31488948:G:A | missense (5/5) | c.1349G > A | p.Gly450Glu | No | No | Yes | No |
| 16:31488952:G:C | missense (5/5) | c.1353G > C | p.Gln451His | No | No | Yes | No |
| 16:31488965:A:T | missense (5/5) | c.1366A > T | p.Ile456Phe | No | No | Yes | No |
| 16:31488966:T:A | missense (5/5) | c.1367T > A | p.Ile456Asn | No | No | Yes | No |
| 16:31488969:A:G | missense (5/5) | c.1370A > G | p.Gln457Arg | No | No | Yes | No |
| 16:31488969:A:C | missense (5/5) | c.1370A > C | p.Gln457Pro | No | No | Yes | No |
| 16:31488970:G:T | missense (5/5) | c.1371G > T | p.Gln457His | No | No | Yes | No |
| 16:31488981:G:C | missense (5/5) | c.1382G > C | p.Ser461Thr | No | No | Yes | No |
| 16:31488983:T:C | missense (5/5) | c.1384T > C | p.Tyr462His | No | No | Yes | No |
| 16:31488983:T:G | missense (5/5) | c.1384T > G | p.Tyr462Asp | No | No | Yes | No |
| 16:31488987:T:C | missense (5/5) | c.1388T > C | p.Leu463Pro | No | No | Yes | No |
| 16:31488993:C:T | missense (5/5) | c.1394C > T | p.Pro465Leu | No | No | Yes | No |
| 16:31488995:C:T | missense (5/5) | c.1396C > T | p.Pro466Ser | No | No | Yes | No |
| 16:31489001:T:C | missense (5/5) | c.1402T > C | p.Ser468Pro | No | No | Yes | No |
| 16:31489004:G:A | missense (5/5) | c.1405G > A | p.Ala469Thr | No | No | Yes | No |
| 16:31489004:GC:G | frameshift | c.1407delC | p.Val470fs | No | No | Yes | Yes |
| 16:31489008:T:C | missense (5/5) | c.1409T > C | p.Val470Ala | No | No | Yes | No |
| 16:31489017:T:G | missense (5/5) | c.1418T > G | p.Leu473Arg | No | No | Yes | No |
| 16:31489017:T:C | missense (5/5) | c.1418T > C | p.Leu473Pro | No | No | Yes | No |
| 16:31489019:G:A | missense (5/5) | c.1420G > A | p.Ala474Thr | No | No | Yes | No |
| 16:31489034:C:T | missense (5/5) | c.1435C > T | p.Arg479Cys | No | No | Yes | No |
| 16:31489040:A:C | missense (5/5) | c.1441A > C | p.Asn481His | No | No | Yes | No |
| 16:31489044:A:G | missense (5/5) | c.1445A > G | p.Glu482Gly | No | No | Yes | No |
| 16:31489045:G:C | missense (5/5) | c.1446G > C | p.Glu482Asp | No | No | Yes | No |
| 16:31489122:G:A | splice acceptor | c.1450 − 1G > A | | No | No | Yes | Yes |
| 16:31489126:G:A | missense (5/5) | c.1453G > A | p.Ala485Thr | No | No | Yes | No |
| 16:31489130:T:C | missense (5/5) | c.1457T > C | p.Phe486Ser | No | No | Yes | No |
| 16:31489134:G:A | stop gained | c.1461G > A | p.Trp487* | No | No | Yes | Yes |
| 16:31489136:G:A | missense (5/5) | c.1463G > A | p.Gly488Glu | No | No | Yes | No |
| 16:31489148:G:T | missense (5/5) | c.1475G > T | p.Gly492Val | No | No | Yes | No |
| 16:31489151:T:G | missense (5/5) | c.1478T > G | p.Leu493Arg | No | No | Yes | No |
| 16:31489159:G:A | missense (5/5) | c.1486G > A | p.Gly496Ser | No | No | Yes | No |
| 16:31489160:G:C | missense (5/5) | c.1487G > C | p.Gly496Ala | No | No | Yes | No |
| 16:31489168:C:T | missense (5/5) | c.1495C > T | p.Arg499Cys | No | No | Yes | No |
| 16:31489180:G:T | stop gained | c.1507G > T | p.Glu503* | No | No | Yes | Yes |
| 16:31489184:T:A | missense (5/5) | c.1511T > A | p.Phe504Tyr | No | No | Yes | No |
| 16:31489193:G:GC | frameshift | c.1521dupc | p.Ser508fs | No | No | Yes | Yes |
| 16:31489198:G:C | missense (5/5) | c.1525G > C | p.Gly509Arg | No | No | Yes | No |
| 16:31489199:G:A | missense (5/5) | c.1526G > A | p.Gly509Asp | No | No | Yes | No |
| 16:31489205:G:C | missense (5/5) | c.1532G > C | p.Cys511Ser | No | No | Yes | No |
| 16:31489205:G:A | missense (5/5) | c.1532G > A | p.Cys511Tyr | No | No | Yes | No |
| 16:31489210:C:T | stop gained | c.1537C > T | p.Gln513* | No | No | Yes | Yes |
| 16:31489225:C:A | missense (5/5) | c.1552C > A | p.Pro518Thr | No | No | Yes | No |
| 16:31489226:C:G | missense (5/5) | c.1553C > G | p.Pro518Arg | No | No | Yes | No |
| 16:31489239:C:A | stop gained | c.1566C > A | p.Cys522* | No | No | Yes | Yes |
| 16:31489246:C:T | missense (5/5) | c.1573C > T | p.His525Tyr | No | No | Yes | No |
| 16:31489248:C:G | missense (5/5) | c.1575C > G | p.His525Gln | No | No | Yes | No |
| 16:31489249:T:C | missense (5/5) | c.1576T > C | p.Tyr526His | No | No | Yes | No |
| 16:31489251:C:A | stop gained | c.1578C > A | p.Tyr526* | No | No | Yes | Yes |
| 16:31489252:C:T | missense (5/5) | c.1579C > T | p.Leu527Phe | No | No | Yes | No |
| 16:31489253:T:C | missense (5/5) | c.1580T > C | p.Leu527Pro | No | No | Yes | No |
| 16:31489256:A:G | missense (5/5) | c.1583A > G | p.Tyr528Cys | No | No | Yes | No |
| 16:31489264:AT:A | frameshift | c.1593delT | p.Ile531fs | No | No | Yes | Yes |
| 16:31489271:T:C | missense (5/5) | c.1598T > C | p.Leu533Pro | No | No | Yes | No |
| 16:31489275:C:G | missense (5/5) | c.1602C > G | p.Phe534Leu | No | No | Yes | No |
| 16:31489279:T:C | missense (5/5) | c.1606T > C | p.Cys536Arg | No | No | Yes | No |
| 16:31489282:T:C | missense (5/5) | c.1609T > C | p.Ser537Pro | No | No | Yes | No |
| 16:31489291:C:T | missense (5/5) | c.1618C > T | p.Leu540Phe | No | No | Yes | No |
| 16:31489307:C:G | missense (5/5) | c.1634C > G | p.Ser545Cys | No | No | Yes | No |
| 16:31489322:C:G | missense (5/5) | c.1649C > G | p.Pro550Arg | No | No | Yes | No |
| 16:31489333:A:T | stop gained | c.1660A > T | p.Lys554* | No | No | Yes | Yes |
| 16:31489338:CG:C | splice donor | c.1665 + 1delG | | No | No | Yes | Yes |
| 16:31489339:G:C | splice donor | c.1665 + 1G > C | | No | No | Yes | Yes |
| 16:31489339:G:A | splice donor | c.1665 + 1G > A | | No | No | Yes | Yes |
| 16:31490103:G:A | splice acceptor | c.1666 − 1G > A | | No | No | Yes | Yes |
| 16:31490104:C:T | missense (5/5) | c.1666C > T | p.Leu556Phe | No | No | Yes | No |
| 16:31490110:C:T | missense (5/5) | c.1672C > T | p.Arg558Cys | No | No | Yes | No |
| 16:31490128:C:T | missense (5/5) | c.1690C > T | p.Arg564Trp | No | No | Yes | No |
| 16:31490129:G:T | missense (5/5) | c.1691G > T | p.Arg564Leu | No | No | Yes | No |
| 16:31490129:G:A | missense (5/5) | c.1691G > A | p.Arg564Gln | No | No | Yes | No |
| 16:31490146:C:T | missense (5/5) | c.1708C > T | p.Arg570Trp | No | No | Yes | No |
| 16:31490207:CAG:C | frameshift | c.1774_1775delAG | p.Ser592fs | No | No | Yes | Yes |
| 16:31490230:G:T | stop gained | c.1792G > T | p.Glu598* | No | No | Yes | Yes |
| 16:31490232:T:C | splice donor | c.1792 + 2T > C | | No | No | Yes | Yes |
| 16:31490362:G:A | missense (5/5) | c.1846G > A | p.Gly616Arg | No | No | Yes | No |
| 16:31490371:AGAGGT:A | frameshift | c.1857_1861delAGGTG | p.Val622fs | No | No | Yes | Yes |
| 16:31490449:G:A | missense (5/5) | c.1933G > A | p.Glu645Lys | No | No | Yes | No |
| 16:31490498:T:C | missense (5/5) | c.1982T > C | p.Met661Thr | No | No | Yes | No |
| 16:31490519:G:A | stop gained | c.2003G > A | p.Trp668* | No | No | Yes | Yes |
| 16:31490520:G:C | missense (5/5) | c.2004G > C | p.Trp668Cys | No | No | Yes | No |
| 16:31490521:G:A | missense (5/5) | c.2005G > A | p.Gly669Ser | No | No | Yes | No |
| 1:62597712:G:A | missense (5/5) | c.146G > A | p.Gly49Asp | Yes | No | No | No |
| 1:62597715:T:G | missense (5/5) | c.149T > G | p.Leu50Arg | Yes | No | No | No |
| 1:62597756:A:T | missense (5/5) | c.190A > T | p.Thr64Ser | Yes | No | No | No |
| 1:62597915:T:G | missense (5/5) | c.349T > G | p.Ser117Ala | Yes | No | No | No |
| 1:62597970:A:G | missense (5/5) | c.404A > G | p.Gln135Arg | Yes | No | No | No |
| 1:62598003:CTAACT:C | frameshift | c.441_445delCTTAA | p.Leu148fs | Yes | Yes | No | No |
| 1:62598700:T:C | missense (5/5) | c.500T > C | p.Phe167Ser | Yes | No | No | No |
| 1:62598775:A:G | missense (5/5) | c.575A > G | p.Gln192Arg | Yes | No | No | No |
| 1:62601132:GC:G | frameshift | c.659delC | p.Pro220fs | Yes | Yes | No | No |
| 1:62601153:T:TC | frameshift | c.681dupC | p.Phe228fs | Yes | Yes | No | No |
| 1:62601168:GA:G | frameshift | c.695delA | p.Asn232fs | Yes | Yes | No | No |
| 1:62601883:G:C | splice donor | c.835 + 1G > C | | Yes | Yes | No | No |
| 1:62602367:TG:T | frameshift | c.921delG | p.Arg308fs | Yes | Yes | No | No |
| 1:62603967:A:G | splice acceptor | c.932 − 2A > G | | Yes | Yes | No | No |
| 1:62604046:G:T | missense (5/5) | c.1009G > T | p.Asp337Tyr | Yes | No | No | No |
| 1:62604691:A:AC | frameshift | c.1259dupc | p.Arg421fs | Yes | Yes | No | No |
| 1:62604767:A:AT | frameshift | c.1333_1334insT | p.Lys445fs | Yes | Yes | No | No |
| 16:31483201:A:G | missense (5/5) | c.65A > G | p.Asn22Ser | No | No | Yes | No |
| 16:31484674:C:A | missense (5/5) | c.128C > A | p.Ser43Tyr | No | No | Yes | No |
| 16:31484736:T:C | missense (5/5) | c.190T > C | p.Trp64Arg | No | No | Yes | No |
| 16:31484818:G:C | splice acceptor | c.199 − 1G > C | | No | No | Yes | Yes |
| 16:31484820:T:A | missense (5/5) | c.200T > A | p.Val67Asp | No | No | Yes | No |
| 16:31484901:C:A | missense (5/5) | c.281C > A | p.Ala94Asp | No | No | Yes | No |
| 16:31485756:T:C | missense (5/5) | c.331T > C | p.Trp111Arg | No | No | Yes | No |
| 16:31485758:G:A | stop gained | c.333G > A | p.Trp111* | No | No | Yes | Yes |
| 16:31485789:G:T | missense (5/5) | c.364G > T | p.Val122Phe | No | No | Yes | No |
| 16:31485871:T:C | missense (5/5) | c.446T > C | p.Leu149Pro | No | No | Yes | No |
| 16:31485894:G:T | splice donor | c.468 + 1G > T | | No | No | Yes | Yes |
| 16:31487322:G:C | missense (5/5) | c.577G > C | p.Gly193Arg | No | No | Yes | No |
| 16:31487324:G:GC | frameshift | c.580dupC | p.Leu194fs | No | No | Yes | Yes |
| 16:31487364:G:T | missense (5/5) | c.619G > T | p.Val207Phe | No | No | Yes | No |
| 16:31487659:C:T | missense (5/5) | c.785C > T | p.Ser262Phe | No | No | Yes | No |
| 16:31487671:T:C | missense (5/5) | c.797T > C | p.Leu266Pro | No | No | Yes | No |
| 16:31487729:C:G | missense (5/5) | c.855C > G | p.Ile285Met | No | No | Yes | No |
| 16:31487748:T:C | missense (5/5) | c.874T > C | p.Cys292Arg | No | No | Yes | No |
| 16:31488044:G:C | missense (5/5) | c.892G > C | p.Val298Leu | No | No | Yes | No |
| 16:31488072:T:C | missense (5/5) | c.920T > C | p.Leu307Pro | No | No | Yes | No |
| 16:31488164:C:G | missense (5/5) | c.1012C > G | p.Leu338Val | No | No | Yes | No |
| 16:31488385:G:T | stop gained | c.1024G > T | p.Glu342* | No | No | Yes | Yes |
| 16:31488386:A:T | missense (5/5) | c.1025A > T | p.Glu342Val | No | No | Yes | No |
| 16:31488388:G:T | missense (5/5) | c.1027G > T | p.Val343Leu | No | No | Yes | No |
| 16:31488443:G:A | missense (5/5) | c.1082G > A | p.Cys361Tyr | No | No | Yes | No |
| 16:31488466:C:T | missense (5/5) | c.1105C > T | p.Leu369Phe | No | No | Yes | No |
| 16:31488642:G:C | missense (5/5) | c.1150G > C | p.Ala384Pro | No | No | Yes | No |
| 16:31488643:C:A | missense (5/5) | c.1151C > A | p.Ala384Glu | No | No | Yes | No |
| 16:31488652:T:C | missense (5/5) | c.1160T > C | p.Leu387Pro | No | No | Yes | No |
| 16:31488654:G:A | missense (5/5) | c.1162G > A | p.Ala388Thr | No | No | Yes | No |
| 16:31488691:G:A | missense (5/5) | c.1199G > A | p.Ser400Asn | No | No | Yes | No |
| 16:31488709:C:T | missense (5/5) | c.1217C > T | p.Thr406Ile | No | No | Yes | No |
| 16:31488741:G:A | missense (5/5) | c.1249G > A | p.Ala417Thr | No | No | Yes | No |
| 16:31488757:T:C | missense (5/5) | c.1265T > C | p.Leu422Pro | No | No | Yes | No |
| 16:31488774:T:G | splice donor | c.1280 + 2T > G | | No | No | Yes | Yes |
| 16:31488879:G:A | splice acceptor | c.1281 − 1G > A | | No | No | Yes | Yes |
| 16:31488915:C:A | missense (5/5) | c.1316C > A | p.Ala439Asp | No | No | Yes | No |
| 16:31488945:G:C | missense (5/5) | c.1346G > C | p.Gly449Ala | No | No | Yes | No |
| 16:31488958:C:A | missense (5/5) | c.1359C > A | p.Phe453Leu | No | No | Yes | No |
| 16:31488976:C:CTCTA | frameshift | c.1378_1381dupTCTA | p.Ser461fs | No | No | Yes | Yes |
| 16:31489142:TC:T | frameshift | c.1470delC | p.Ile490fs | No | No | Yes | Yes |
| 16:31489145:G:A | missense (5/5) | c.1472G > A | p.Gly491Glu | No | No | Yes | No |
| 16:31489163:T:C | missense (5/5) | c.1490T > C | p.Leu497Pro | No | No | Yes | No |
| 16:31489166:C:A | missense (5/5) | c.1493C > A | p.Ala498Glu | No | No | Yes | No |
| 16:31489249:T:A | missense (5/5) | c.1576T > A | p.Tyr526Asn | No | No | Yes | No |
| 16:31489250:A:G | missense (5/5) | c.1577A > G | p.Tyr526Cys | No | No | Yes | No |
| 16:31489264:A:T | missense (5/5) | c.1591A > T | p.Ile531Phe | No | No | Yes | No |
| 16:31489270:CTG:C | frameshift | c.1599_1600delGT | p.Phe534fs | No | No | Yes | Yes |
| 16:31489275:C:CTT | frameshift | c.1603_1604dupTT | p.Cys536fs | No | No | Yes | Yes |
| 16:31490231:G:C | splice donor | c.1792 + 1G > C | | No | No | Yes | Yes |
| 1:62597596:TG:T | frameshift | c.31delG | p.Val11fs | Yes | Yes | No | No |
| 1:62597674:A:T | missense (5/5) | c.108A > T | p.Arg36Ser | Yes | No | No | No |
| 1:62597685:T:C | missense (5/5) | c.119T > C | p.Leu40Ser | Yes | No | No | No |
| 1:62597691:A:T | missense (5/5) | c.125A > T | p.Asp42Val | Yes | No | No | No |
| 1:62597715:T:A | missense (5/5) | c.149T > A | p.Leu50His | Yes | No | No | No |
| 1:62597717:C:G | missense (5/5) | c.151C > G | p.Leu51Val | Yes | No | No | No |
| 1:62597736:T:A | missense (5/5) | c.170T > A | p.Leu57His | Yes | No | No | No |
| 1:62597736:T:G | missense (5/5) | c.170T > G | p.Leu57Arg | Yes | No | No | No |
| 1:62597747:G:A | missense (5/5) | c.181G > A | p.Val61Ile | Yes | No | No | No |
| 1:62597754:A:T | missense (5/5) | c.188A > T | p.Lys63Met | Yes | No | No | No |
| 1:62597757:C:A | missense (5/5) | c.191C > A | p.Thr64Lys | Yes | No | No | No |
| 1:62597766:A:C | missense (5/5) | c.200A > C | p.Gln67Pro | Yes | No | No | No |
| 1:62597801:G:T | missense (5/5) | c.235G > T | p.Asp79Tyr | Yes | No | No | No |
| 1:62597885:C:T | stop gained | c.319C > T | p.Gln107* | Yes | Yes | No | No |
| 1:62598753:C:T | stop gained | c.553C > T | p.Gln185* | Yes | Yes | No | No |
| 1:62598807:G:T | splice donor | c.606 + 1G > T | | Yes | Yes | No | No |
| 1:62601092:CT:C | frameshift | c.618delT | p.Ser207fs | Yes | Yes | No | No |
| 1:62601197:G:T | splice donor | c.721 + 1G > T | | Yes | Yes | No | No |
| 1:62601198:T:C | splice donor | c.721 + 2T > c | | Yes | Yes | No | No |
| 1:62601783:T:C | missense (5/5) | c.736T > C | p.Cys246Arg | Yes | No | No | No |
| 1:62601802:G:GAGGT | frameshift | c.756_759dupAGGT | p.Glu254fs | Yes | Yes | No | No |
| 1:62601866:CTG:C | frameshift | c.822_823delTG | p.Cys274fs | Yes | Yes | No | No |
| 1:62602295:G:A | stop gained | c.846G > A | p.Trp282* | Yes | Yes | No | No |
| 1:62602297:C:CA | frameshift | c.849dupA | p.Leu284fs | Yes | Yes | No | No |
| 1:62602305:C:A | missense (5/5) | c.856C > A | p.Gln286Lys | Yes | No | No | No |
| 1:62602332:T:C | missense (5/5) | c.883T > C | p.Phe295Leu | Yes | No | No | No |
| 1:62602382:T:C | splice donor | c.931 + 2T > C | | Yes | Yes | No | No |
| 1:62604005:T:G | missense (5/5) | c.968T > G | p.Ile323Arg | Yes | No | No | No |
| 1:62604173:T:G | missense (5/5) | c.1136T > G | p.Leu379Trp | Yes | No | No | No |
| 1:62604174:G:C | missense (5/5) | c.1137G > C | p.Leu379Phe | Yes | No | No | No |
| 1:62604174:GGT:G | frameshift | c.1140_1141delGT | p.Ser382fs | Yes | Yes | No | No |
| 1:62604185:C:G | missense (5/5) | c.1148C > G | p.Thr383Ser | Yes | No | No | No |
| 1:62604224:AG:A | frameshift | c.1190delG | p.Gly397fs | Yes | Yes | No | No |
| 1:62604635:G:A | missense (5/5) | c.1201G > A | p.Gly401Ser | Yes | No | No | No |
| 1:62604643:G:C | missense (5/5) | c.1209G > C | p.Trp403Cys | Yes | No | No | No |
| 1:62604685:T:A | stop gained | c.1251T > A | p.Tyr417* | Yes | Yes | No | No |
| 1:62604726:G:A | missense (5/5) | c.1292G > A | p.Gly431Glu | Yes | No | No | No |
| 1:62604734:TG:T | frameshift | c.1302delG | p.Trp434fs | Yes | Yes | No | No |
| 1:62604735:G:T | missense (5/5) | c.1301G > T | p.Trp434Leu | Yes | No | No | No |
| 1:62604737:A:G | missense (5/5) | c.1303A > G | p.Lys435Glu | Yes | No | No | No |
| 1:62604768:A:G | missense (5/5) | c.1334A > G | p.Lys445Arg | Yes | No | No | No |
| 1:62604780:T:C | missense (5/5) | c.1346T > C | p.Met449Thr | Yes | No | No | No |
| 1:62604815:T:G | stop lost | c.1381T > G | p.Ter461Glyext*? | Yes | Yes | No | No |
| 16:31483138:T:G | start lost | c.2T > G | p.Met1? | No | No | Yes | Yes |
| 16:31483219:T:C | missense (5/5) | c.83T > C | p.Val28Ala | No | No | Yes | No |
| 16:31483248:G:C | missense (5/5) | c.112G > C | p.Gly38Arg | No | No | Yes | No |
| 16:31484732:G:C | missense (5/5) | c.186G > C | p.Met62Ile | No | No | Yes | No |
| 16:31484735:G:GTGGTGGC | frameshift | c.190_196dupTGGTGGC | p.Pro66fs | No | No | Yes | Yes |
| 16:31484737:G:A | stop gained | c.191G > A | p.Trp64* | No | No | Yes | Yes |
| 16:31484743:C:G | missense (5/5) | c.197C > G | p.Pro66Arg | No | No | Yes | No |
| 16:31484833:CTT:C | frameshift | c.214_215delTT | p.Phe72fs | No | No | Yes | Yes |
| 16:31484847:T:C | missense (5/5) | c.227T > C | p.Ile76Thr | No | No | Yes | No |
| 16:31484860:CT:C | frameshift | c.243delT | p.Phe81fs | No | No | Yes | Yes |
| 16:31484873:G:A | missense (5/5) | c.253G > A | p.Ala85Thr | No | No | Yes | No |
| 16:31484901:C:T | missense (5/5) | c.281C > T | p.Ala94Val | No | No | Yes | No |
| 16:31484914:C:G | missense (5/5) | c.294C > G | p.Phe98Leu | No | No | Yes | No |
| 16:31484915:G:C | missense (5/5) | c.295G > C | p.Glu99Gln | No | No | Yes | No |
| 16:31485771:G:C | missense (5/5) | c.346G > C | p.Val116Leu | No | No | Yes | No |
| 16:31485780:ACAGCGGGGGTCAT:A | frameshift | c.358_370delGCGGGGGTCATCA | p.Ala120fs | No | No | Yes | Yes |
| 16:31485796:C:G | missense (5/5) | c.371C > G | p.Thr124Arg | No | No | Yes | No |
| 16:31485849:C:G | missense (5/5) | c.424C > G | p.Leu142Val | No | No | Yes | No |
| 16:31485892:C:T | missense (5/5) | c.467C > T | p.Ser156Leu | No | No | Yes | No |
| 16:31486260:AT:A | frameshift | c.562delT | p.Tyr188fs | No | No | Yes | Yes |
| 16:31486261:T:TGC | frameshift | c.560_561insGC | p.Ile187fs | No | No | Yes | Yes |
| 16:31486263:T:G | missense (5/5) | c.562T > G | p.Tyr188Asp | No | No | Yes | No |
| 16:31486264:A:G | missense (5/5) | c.563A > G | p.Tyr188Cys | No | No | Yes | No |
| 16:31487346:G:A | missense (5/5) | c.601G > A | p.Asp201Asn | No | No | Yes | No |
| 16:31487350:C:T | missense (5/5) | c.605C > T | p.Thr202Met | No | No | Yes | No |
| 16:31487400:G:A | missense (5/5) | c.655G > A | p.Ala219Thr | No | No | Yes | No |
| 16:31487538:G:T | stop gained | c.664G > T | p.Glu222* | No | No | Yes | Yes |
| 16:31487541:G:A | missense (5/5) | c.667G > A | p.Val223Met | No | No | Yes | No |
| 16:31487542:TG:T | frameshift | c.671delG | p.Gly224fs | No | No | Yes | Yes |
| 16:31487647:C:G | missense (5/5) | c.773C > G | p.Pro258Arg | No | No | Yes | No |
| 16:31487719:G:A | missense (5/5) | c.845G > A | p.Gly282Glu | No | No | Yes | No |
| 16:31487746:G:A | stop gained | c.872G > A | p.Trp291* | No | No | Yes | Yes |
| 16:31487757:C:T | stop gained | c.883C > T | p.Gln295* | No | No | Yes | Yes |
| 16:31487758:A:C | missense (5/5) | c.884A > C | p.Gln295Pro | No | No | Yes | No |
| 16:31488034:GTA:G | splice acceptor | c.886 − 3_886 − 2delTA | | No | No | Yes | Yes |
| 16:31488044:G:T | missense (5/5) | c.892G > T | p.Val298Leu | No | No | Yes | No |
| 16:31488062:G:T | missense (5/5) | c.910G > T | p.Gly304Trp | No | No | Yes | No |
| 16:31488063:G:A | missense (5/5) | c.911G > A | p.Gly304Glu | No | No | Yes | No |
| 16:31488084:A:G | missense (5/5) | c.932A > G | p.Lys311Arg | No | No | Yes | No |
| 16:31488099:T:C | missense (5/5) | c.947T > C | p.Leu316Pro | No | No | Yes | No |
| 16:31488135:T:A | missense (5/5) | c.983T > A | p.Met328Lys | No | No | Yes | No |
| 16:31488153:T:C | missense (5/5) | c.1001T > C | p.Ile334Thr | No | No | Yes | No |
| 16:31488382:G:A | splice acceptor | c.1022 − 1G > A | | No | No | Yes | Yes |
| 16:31488427:G:C | missense (5/5) | c.1066G > C | p.Gly356Arg | No | No | Yes | No |
| 16:31488442:T:G | missense (5/5) | c.1081T > G | p.Cys361Gly | No | No | Yes | No |
| 16:31488450:C:A | missense (5/5) | c.1089C > A | p.Asn363Lys | No | No | Yes | No |
| 16:31488633:C:T | missense (5/5) | c.1141C > T | p.Leu381Phe | No | No | Yes | No |
| 16:31488642:G:A | missense (5/5) | c.1150G > A | p.Ala384Thr | No | No | Yes | No |
| 16:31488652:T:A | missense (5/5) | c.1160T > A | p.Leu387Gln | No | No | Yes | No |
| 16:31488690:A:G | missense (5/5) | c.1198A > G | p.Ser400Gly | No | No | Yes | No |
| 16:31488692:C:A | missense (5/5) | c.1200C > A | p.Ser400Arg | No | No | Yes | No |
| 16:31488717:AT:A | frameshift | c.1226delT | p.Ile409fs | No | No | Yes | Yes |
| 16:31488720:T:C | missense (5/5) | c.1228T > C | p.Tyr410His | No | No | Yes | No |
| 16:31488721:ACACGCGCCTGCGGC:A | frameshift | c.1238_1251delTGCGGCCACGCGCC | p.Leu413fs | No | No | Yes | Yes |
| 16:31488733:G:T | missense (5/5) | c.1241G > T | p.Arg414Leu | No | No | Yes | No |
| 16:31488772:G:C | missense (5/5) | c.1280G > C | p.Arg427Pro | No | No | Yes | No |
| 16:31488774:T:C | splice donor | c.1280 + 2T > C | | No | No | Yes | Yes |
| 16:31488888:TG:T | frameshift | c.1291delG | p.Val431fs | No | No | Yes | Yes |
| 16:31488900:TG:T | frameshift | c.1303delG | p.Val435fs | No | No | Yes | Yes |
| 16:31488917:T:G | missense (5/5) | c.1318T > G | p.Trp440Gly | No | No | Yes | No |
| 16:31488930:T:C | missense (5/5) | c.1331T > C | p.Val444Ala | No | No | Yes | No |
| 16:31488948:G:T | missense (5/5) | c.1349G > T | p.Gly450Val | No | No | Yes | No |
| 16:31488957:T:G | missense (5/5) | c.1358T > G | p.Phe453Cys | No | No | Yes | No |
| 16:31488982:C:G | missense (5/5) | c.1383C > G | p.Ser461Arg | No | No | Yes | No |
| 16:31488992:C:G | missense (5/5) | c.1393C > G | p.Pro465Ala | No | No | Yes | No |
| 16:31489022:C:T | missense (5/5) | c.1423C > T | p.Leu475Phe | No | No | Yes | No |
| 16:31489042:T:A | missense (5/5) | c.1443T > A | p.Asn481Lys | No | No | Yes | No |
| 16:31489123:G:C | missense (5/5) | c.1450G > C | p.Gly484Arg | No | No | Yes | No |
| 16:31489124:G:C | missense (5/5) | c.1451G > C | p.Gly484Ala | No | No | Yes | No |
| 16:31489133:G:C | missense (5/5) | c.1460G > C | p.Trp487Ser | No | No | Yes | No |
| 16:31489148:G:A | missense (5/5) | c.1475G > A | p.Gly492Asp | No | No | Yes | No |
| 16:31489151:T:C | missense (5/5) | c.1478T > C | p.Leu493Pro | No | No | Yes | No |
| 16:31489227:AGCTTTCCTCTGCGGCGTGCACT:A | frameshift | c.1555_1576delGCTTTCCTCTGCGGCGTGCACT | p.Ala519fs | No | No | Yes | Yes |
| 16:31489247:A:G | missense (5/5) | c.1574A > G | p.His525Arg | No | No | Yes | No |
| 16:31490146:CG:C | frameshift | c.1711delG | p.Glu571fs | No | No | Yes | Yes |
| 16:31490306:C:CA | splice acceptor | c.1793 − 2dupA | | No | No | Yes | Yes |
| 16:31490307:A:G | splice acceptor | c.1793 − 2A > G | | No | No | Yes | Yes |
| 16:31490346:C:A | stop gained | c.1830C > A | p.Cys610* | No | No | Yes | Yes |
| 16:31490462:G:A | stop gained | c.1946G > A | p.Trp649* | No | No | Yes | Yes |
| 16:31490463:G:A | stop gained | c.1947G > A | p.Trp649* | No | No | Yes | Yes |
| 16:31490518:TG:T | frameshift | c.2006delG | p.Gly669fs | No | No | Yes | Yes |
| 16:31490520:G:A | stop gained | c.2004G > A | p.Trp668* | No | No | Yes | Yes |
| 1:62597678:G:C | missense (5/5) | c.112G > C | p.Ala38Pro | Yes | No | No | No |
| 1:62597741:G:C | missense (5/5) | c.175G > C | p.Asp59His | Yes | No | No | No |
| 1:62597808:CT:C | frameshift | c.247delT | p.Tyr83fs | Yes | Yes | No | No |
| 1:62597936:C:CT | frameshift | c.372dupT | p.Glu125fs | Yes | Yes | No | No |
| 1:62598003:C:CT | frameshift | c.438dupT | p.Asn147fs | Yes | Yes | No | No |
| 1:62598712:A:G | missense (5/5) | c.512A > G | p.Gln171Arg | Yes | No | No | No |
| 1:62601817:G:C | missense (5/5) | c.770G > C | p.Ser257Thr | Yes | No | No | No |
| 1:62602311:C:G | missense (5/5) | c.862C > G | p.Arg288Gly | Yes | No | No | No |
| 1:62602341:AC:A | frameshift | c.893delC | p.Thr298fs | Yes | Yes | No | No |
| 1:62604014:A:T | missense (5/5) | c.977A > T | p.Gln326Leu | Yes | No | No | No |
| 1:62604073:G:T | stop gained | c.1036G > T | p.Glu346* | Yes | Yes | No | No |
| 1:62604078:T:G | stop gained | c.1041T > G | p.Tyr347* | Yes | Yes | No | No |
| 1:62604116:T:C | missense (5/5) | c.1079T > C | p.Leu360Pro | Yes | No | No | No |
| 1:62604672:TAA:T | frameshift | c.1240_1241delAA | p.Asn414fs | Yes | Yes | No | No |
| 16:31484736:T:G | missense (5/5) | c.190T > G | p.Trp64Gly | No | No | Yes | No |
| 16:31485801:C:T | missense (5/5) | c.376C > T | p.Pro126Ser | No | No | Yes | No |
| 16:31485804:C:T | stop gained | c.379C > T | p.Gln127* | No | No | Yes | Yes |
| 16:31485810:C:G | missense (5/5) | c.385C > G | p.Leu129Val | No | No | Yes | No |
| 16:31486213:G:T | missense (5/5) | c.512G > T | p.Gly171Val | No | No | Yes | No |
| 16:31486252:T:C | missense (5/5) | c.551T > C | p.Ile184Thr | No | No | Yes | No |
| 16:31486266:A:G | missense (5/5) | c.565A > G | p.Thr189Ala | No | No | Yes | No |
| 16:31487331:G:A | missense (5/5) | c.586G > A | p.Ala196Thr | No | No | Yes | No |
| 16:31487338:T:G | missense (5/5) | c.593T > G | p.Met198Arg | No | No | Yes | No |
| 16:31487638:G:A | missense (5/5) | c.764G > A | p.Cys255Tyr | No | No | Yes | No |
| 16:31487719:G:GACTC | frameshift | c.848_851dupTCAC | p.Ile285fs | No | No | Yes | Yes |
| 16:31487728:T:C | missense (5/5) | c.854T > C | p.Ile285Thr | No | No | Yes | No |
| 16:31488089:G:C | missense (5/5) | c.937G > C | p.Gly313Arg | No | No | Yes | No |
| 16:31488104:G:C | missense (5/5) | c.952G > C | p.Gly318Arg | No | No | Yes | No |
| 16:31488143:C:T | missense (5/5) | c.991C > T | p.Pro331Ser | No | No | Yes | No |
| 16:31488173:G:C | missense (5/5) | c.1021G > C | p.Asp341His | No | No | Yes | No |
| 16:31488492:T:C | splice donor | c.1129 + 2T > C | | No | No | Yes | Yes |
| 16:31488643:CGGTCATGCTG:C | frameshift | c.1153_1162delGTCATGCTGG | p.Val385fs | No | No | Yes | Yes |
| 16:31488730:TGCGGCCACGCGCCG:T | frameshift | c.1242_1255delGCCACGCGCCGGCG | p.Gly418fs | No | No | Yes | Yes |
| 16:31488757:T:G | missense (5/5) | c.1265T > G | p.Leu422Arg | No | No | Yes | No |
| 16:31488765:G:T | missense (5/5) | c.1273G > T | p.Val425Leu | No | No | Yes | No |
| 16:31488922:TC:T | frameshift | c.1326delC | p.Val443fs | No | No | Yes | Yes |
| 16:31488923:C:A | missense (5/5) | c.1324C > A | p.Pro442Thr | No | No | Yes | No |
| 16:31488992:C:T | missense (5/5) | c.1393C > T | p.Pro465Ser | No | No | Yes | No |
| 16:31488993:C:G | missense (5/5) | c.1394C > G | p.Pro465Arg | No | No | Yes | No |
| 16:31489008:T:A | missense (5/5) | c.1409T > A | p.Val470Asp | No | No | Yes | No |
| 16:31489041:A:G | missense (5/5) | c.1442A > G | p.Asn481Ser | No | No | Yes | No |
| 16:31489217:C:T | missense (5/5) | c.1544C > T | p.Ser515Leu | No | No | Yes | No |
| 16:31489339:G:T | splice donor | c.1665 + 1G > T | | No | No | Yes | Yes |
| 16:31490354:G:C | missense (5/5) | c.1838G > C | p.Trp613Ser | No | No | Yes | No |
| 16:31490360:G:C | missense (5/5) | c.1844G > C | p.Cys615Ser | No | No | Yes | No |
| 16:31490437:G:T | stop gained | c.1921G > T | p.Glu641* | No | No | Yes | Yes |
| 16:31490525:T:C | missense (5/5) | c.2009T > C | p.Phe670Ser | No | No | Yes | No |
| 16:31490530:GC:G | frameshift | c.2016delC | p.Ter673fs | No | No | Yes | Yes |

C:P:R:A indicates the genomic coordinates of the genetic variant including chromosome (C), physical genomic position in base pairs (P), reference allele (R) and alternative allele (A) relative to build 38 of the Human Genome sequence by the Human Genome Reference Consortium. Coding DNA and protein changes follow the Human Genome Variation Society nomenclature and refer to ANGPTL3 or SLC5A2 transcript (ENST00000371129 or ENST00000330498, respectively), annotated in the in the Ensembl database (see, world-wide web at “useast.ensembl.org/index.html”). ‘Missense (5/5)’ and ‘5/5 ms’ indicate missense variants predicted to be damaging by 5 in silico algorithms. pLOF indicates predicted loss of function variant. The right four columns indicate whether the variant was included in gene burden grouping specified, with alternate aIlele frequency (AAF) thresholds <1% for pLOF variants and <0.1% for pLOF or 5/5 missense variants.

### Example 4: General Methods

### Participating Cohorts

The UKB is a population-based cohort study of people aged between 40 and 69 years recruited through 22 testing centers in the UK between 2006-2010 (Sudlow et al., PLoS Med, 2015. 12, e1001779). A total of 431,835 participants from UKB with available whole-exome sequencing and phenotype data were included. The MyCode Community Health Initiative cohort from the Geisinger Health System (GHS) (Carey et al., Genet. Med., 2016, 18, 906-913) is a health system-based cohort of patients from Central and Eastern Pennsylvania (USA) recruited in 2007-2019. A total of 156,846 participants from GHS with available whole-exome sequencing and phenotype data were included. The Mount Sinai's BioMe Personalized Medicine Cohort (SINAI) (Gottesman et al., Genet. Med., 2013, 15, 761-771) is an electronic health record-linked clinical care cohort of 25,839 individuals with available whole-exome sequencing and phenotype data. The University of Pennsylvania Medicine BioBank (UPENN-PMBB) is a health system-based cohort based in Pennsylvania BioBank (Park et al., Nat. Med., 2021, 27, 66-72). The Malmö Diet and Cancer Study (MDCS) is a cohort study based in Malmo, Sweden (Berglund et al., J. Intern. Med., 1993, 233, 45-51). The MCPS is a prospective, population-based study based in Mexico (Tapia-Conyer et al., Int. J. Epidemiol., 2006 35, 243-49).

### Phenotype Definitions

eGFR was calculated from clinical laboratory measurements for creatinine extracted from electronic health records (EHRs) of participants from GHS, SINAI and UPENN-PMBB. Median values were calculated for all participants with two or more measurements. In UKB, eGFR was calculated from creatinine measured on a Beckman Coulter AU5800 clinical chemistry analyzer and cystatin measured by immunoturbidimetric analysis on a Siemens Advia 1800 clinical chemistry analyzer; both creatinine and cystatin were measured at the baseline visit of the study. In MCPS, eGFR was calculated from creatinine measured via nucleic magnetic resonance spectroscopy and converted into clinical units as previously described (Aguilar-Ramirez et al., J. Clin. Endo. Met., 2021, 106, 2828-2839). The Chronic Kidney Disease Epidemiology Collaboration equations for creatinine and cystatin (UKB) or creatinine (all other cohorts) were used to calculate eGFR. Prior to genetic association analysis, eGFR values were transformed by the inverse standard normal function, applied within each ancestry group and separately in men and women.

### Genotype Data

High coverage whole exome sequencing was performed as previously described (Science, 2021, 373:abf8683; and Nature, 2020, 586:749-756) and as summarized below. NimbleGen probes (VCRome; for part of the GHS cohort) or a modified version of the xGen design available from Integrated DNA Technologies (IDT; for the rest of GHS and other cohorts) were used for target sequence capture of the exome. A unique 6 base pair (bp) barcode (VCRome) or 10 bp barcode (IDT) was added to each DNA fragment during library preparation to facilitate multiplexed exome capture and sequencing. Equal amounts of sample were pooled prior to exome capture. Sequencing was performed using 75 bp paired-end reads on Illumina v4 HiSeq 2500 (for part of the GHS cohort) or NovaSeq (for the rest of GHS and other cohorts) instruments. Sequencing had a coverage depth (i.e., number of sequence-reads covering each nucleotide in the target areas of the genome) sufficient to provide greater than 20× coverage over 85% of targeted bases in 96% of VCRome samples and 20× coverage over 90% of targeted bases in 99% of IDT samples. Data processing steps included sample de-multiplexing using Illumina software, alignment to the GRCh38 Human Genome reference sequence including generation of binary alignment and mapping files (BAM), processing of BAM files (e.g., marking of duplicate reads and other read mapping evaluations). Variant calling was performed using the GLNexus system (DOI: 10.1101/343970). Variant mapping and annotation were based on the GRCh38 Human Genome reference sequence and Ensembl v85 gene definitions using the snpEff software. The snpEff predictions that involve protein-coding transcripts with an annotated start and stop were then combined into a single functional impact prediction by selecting the most deleterious functional effect class for each gene. The hierarchy (from most to least deleterious) for these annotations was frameshift, stop-gain, stop-loss, splice acceptor, splice donor, stop-lost, in-frame indel, missense, other annotations. Predicted LOF genetic variants included: a) insertions or deletions resulting in a frameshift, b) insertions, deletions or single nucleotide variants resulting in the introduction of a premature stop codon or in the loss of the transcription start site or stop site, and c) variants in donor or acceptor splice sites. Missense variants were classified for likely functional impact according to the number of in silico prediction algorithms that predicted deleteriousness using SIFT (Adzhubei et al., Nat. Methods, 2010, 7, 248-9) and Polyphen2_HVAR (Adzhubei et al., Nat. Methods, 2010, 7, 248-9), LRT (Chun et al., Genome Res., 2009, 19, 1553-61) and MutationTaster (Schwarz et al., Nat. Methods, 2010, 7, 575-6). For each gene, the alternative allele frequency (AAF) and functional annotation of each variant determined inclusion into 7 gene burden exposures: 1) pLOF variants with AAF<1%; 2) pLOF or missense variants predicted deleterious by 5/5 algorithms with AAF<1%; 3) pLOF or missense variants predicted deleterious by 5/5 algorithms with AAF<0.1%; 4) pLOF or missense variants predicted deleterious by at least 1/5 algorithms with AAF<1%; 5) pLOF or missense variants predicted deleterious by at least 1/5 algorithms with AAF<0.1%; 6) pLOF or any missense with AAF<1%; 7) pLOF or any missense variants with AAF<0.1%.

Association analysis of gene burden of rare pLOF and missense variations We tested for association between the burden of rare predicted loss-of-function or missense variants in a given gene and eGFR by fitting a linear regression model adjusted for a polygenic score that approximates a genomic kinship matrix using REGENIE (Mbatchou et al., Nat. Genetics, 2021, 53, 1097-1103). Analyses were stratified by ancestry and adjusted for age, age², sex, age-by-sex and age²-by-sex interaction terms, experimental batch-related covariates, 10 common variant-derived principal components, and 20 rare variant-derived principal components. Results across cohorts for each variant-phenotype association were combined using fixed effects inverse variance weighted meta-analysis. In gene burden tests, all individuals are labeled as heterozygotes if they carry one or more qualifying rare variant (as described above based on frequency and functional annotation) and as homozygotes if they carry any qualifying variant in the homozygous state. This “composite genotype” is then used to test for association. The burden exposure with the strongest association with eGFR as well as the burden of pLOF variants with AAF<1% were taken forward for further analysis.

To examine whether the effects of ANGPTL3 gene burdens on eGFR varied depending on SLC5A2 gene burdens and vice versa, we took two approaches. First, we adjusted for the second gene burden by including it as a covariate in the linear regression model between the first gene burden and eGFR. We compared these results to those from the linear regression model between the first gene burden and eGFR, without adjustment for the second gene burden. Second, we performed a formal interaction analysis of eGFR using a linear regression model fit with the ANGPTL3 gene burden, the SLC5A2 gene burden, and an interaction term of ANGPTL3*SLC5A2 gene burdens. The p-value of interaction term allows assessment of whether the effect of the ANGPTL3 gene burden is significantly different among different genotypes of the SLC5A2 gene burden (and vice versa). Both analyses were performed as described in the previous paragraph, substituting a modified REGENIE polygenic score that excluded both chromosomes containing the ANGPTL3 and SLC5A2 genes.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a kidney disease or at risk of developing a kidney disease, the method comprising administering an Angiopoietin Like 3 (ANGPTL3) inhibitor and a Solute Carrier Family 5 Member 2 (SLC5A2) inhibitor to the subject.

2. The method of claim 1, wherein the kidney disease is chronic kidney disease, a diabetic kidney disease, chronic glomerulonephritis, nephronophthisis, chronic interstitial nephritis, or nephrosclerosis.

3-7. (canceled)

8. The method of claim 1, wherein the ANGPTL3 inhibitor comprises an inhibitory nucleic acid molecule that hybridizes to an ANGPTL3 nucleic acid molecule.

9. The method of claim 8, wherein the inhibitory nucleic acid molecule comprises an antisense nucleic acid molecule, a small interfering RNA (siRNA), or a short hairpin RNA (shRNA).

10-12. (canceled)

13. The method of claim 1, wherein the ANGPTL3 inhibitor comprises an antibody.

14. The method of claim 13, wherein the antibody comprises evinacumab.

15. The method of claim 1, wherein the SLC5A2 inhibitor comprises an inhibitory nucleic acid molecule that hybridizes to a SLC5A2 nucleic acid molecule.

16. The method of claim 15, wherein the inhibitory nucleic acid molecule comprises an antisense nucleic acid molecule, a small interfering RNA (siRNA), or a short hairpin RNA (shRNA).

17-18. (canceled)

19. The method of claim 1, wherein the SLC5A2 inhibitor comprises canagliflozin, dapagliflozin, empagliflozin, ipragliflozin, luseogliflozin, or tofogliflozin, or any combination thereof.

20. The method of claim 1, further comprising detecting the presence or absence of an ANGPTL3 variant nucleic acid molecule and detecting the presence or absence of an SLC5A2 variant nucleic acid molecule in a biological sample from the subject.

21. The method of claim 20, wherein the ANGPTL3 variant nucleic acid molecule is a missense variant, a splice-site variant, a stop-gain variant, a start-loss variant, a stop-loss variant, a frameshift variant, an in-frame indel variant, or a variant that encodes a truncated ANGPTL3 polypeptide.

22. The method of claim 20, wherein the SLC5A2 variant nucleic acid molecule is a missense variant, a splice-site variant, a stop-gain variant, a start-loss variant, a stop-loss variant, a frameshift variant, an in-frame indel variant, or a variant that encodes a truncated SLC5A2 polypeptide.

23. The method of claim 20, further comprising administering a kidney disease therapeutic agent to a subject that is: i) ANGPTL3 reference or heterozygous for the ANGPTL3 variant nucleic acid molecule, and ii) SLC5A2 reference or heterozygous for the SLC5A2 variant nucleic acid molecule.

24. A method of treating a subject with a kidney disease therapeutic agent, wherein the subject has a kidney disease or is at risk of developing a kidney disease, by administering a kidney disease therapeutic agent, the method comprising:
determining whether the subject has an Angiopoietin Like 3 (ANGPTL3) variant nucleic acid molecule and whether the subject has a Solute Carrier Family 5 Member 2 (SLC5A2) variant nucleic acid molecule, by:
obtaining or having obtained a biological sample from the subject; and
performing or having performed a sequence analysis on the biological sample to determine if the subject has a genotype comprising: i) the ANGPTL3 variant nucleic acid molecule, and ii) the SLC5A2 variant nucleic acid molecule; and
administering or continuing to administer the kidney disease therapeutic agent to a subject that is homozygous for both the ANGPTL3 variant nucleic acid molecule and the SLC5A2 variant nucleic acid molecule; or
administering or continuing to administer the kidney disease therapeutic agent and/or an ANGPTL3 inhibitor and an SLC5A2 inhibitor to a subject that is: i) heterozygous for both the ANGPTL3 variant nucleic acid molecule and the SLC5A2 variant nucleic acid molecule, ii) heterozygous for one of the ANGPTL3 variant nucleic acid molecule and the SLC5A2 variant nucleic acid molecule and reference for the other of the ANGPTL3 variant nucleic acid molecule and the SLC5A2 variant nucleic acid molecule, or iii) ANGPTL3 reference and SLC5A2 reference;
wherein the presence of a genotype having the ANGPTL3 variant nucleic acid molecule and the SLC5A2 variant nucleic acid molecule indicates the subject has a decreased risk of developing a kidney disease.

25. The method of claim 24, wherein the kidney disease is chronic kidney disease, diabetic kidney disease, chronic glomerulonephritis, nephronophthisis, chronic interstitial nephritis, or nephrosclerosis.

26-30. (canceled)

31. The method of claim 24, wherein the ANGPTL3 variant nucleic acid molecule is a missense variant, a splice-site variant, a stop-gain variant, a start-loss variant, a stop-loss variant, a frameshift variant, an in-frame indel variant, or a variant that encodes a truncated ANGPTL3 polypeptide.

32. The method of claim 24, wherein the SLC5A2 variant nucleic acid molecule is a missense variant, a splice-site variant, a stop-gain variant, a start-loss variant, a stop-loss variant, a frameshift variant, an in-frame indel variant, or a variant that encodes a truncated SLC5A2 polypeptide.

33. The method of claim 24, wherein the subject is: i) ANGPTL3 reference or heterozygous for the ANGPTL3 variant nucleic acid molecule, and ii) SLC5A2 reference or heterozygous for the SLC5A2 variant nucleic acid molecule, and the subject is administered or continued to be administered the kidney disease therapeutic agent and/or an ANGPTL3 inhibitor and an SLC5A2 inhibitor.

34. The method of claim 24, wherein the ANGPTL3 inhibitor comprises an inhibitory nucleic acid molecule that hybridizes to an ANGPTL3 nucleic acid molecule.

35. The method of claim 34, wherein the inhibitory nucleic acid molecule comprises an antisense nucleic acid molecule, a small interfering RNA (siRNA), or a short hairpin RNA (shRNA).

36-38. (canceled)

39. The method of claim 24, wherein the ANGPTL3 inhibitor comprises an antibody.

40. The method of claim 39, wherein the antibody comprises evinacumab.

41. The method of claim 24, wherein the SLC5A2 inhibitor comprises an inhibitory nucleic acid molecule that hybridizes to a SLC5A2 nucleic acid molecule.

42. The method of claim 41, wherein the inhibitory nucleic acid molecule comprises an antisense nucleic acid molecule, a small interfering RNA (siRNA), or a short hairpin RNA (shRNA).

43-44. (canceled)

45. The method of claim 24, wherein the SLC5A2 inhibitor comprises canagliflozin, dapagliflozin, empagliflozin, ipragliflozin, luseogliflozin, or tofogliflozin, or any combination thereof.

46-85. (canceled)
